(19) United States
(12) Patent Application Publication
(10) Pub. No.: US 2012/0090278 A1
(43) Pub. Date: Apr. 19, 2012

(54) DISPOSABLE TRANSDERMAL PATCH CONTAINMENT

(75) Inventors: Vernon D. Ortenzi, Burlington, KY (US); Robert J. Ziemba, Cincinnati, OH (US); James A. Topich, Mason, OH (US)

(73) Assignee: MALLINCKRODT LLC, Hazelwood, MO (US)

(21) Appl. No.: 13/272,904

(22) Filed: Oct. 13, 2011

Related U.S. Application Data
(60) Provisional application No. 61/392,547, filed on Oct. 13, 2010.

(51) Int. Cl. B65B 5/10; A61B 19/02

(52) U.S. Cl. 53/475; 206/438

(57) ABSTRACT

Various embodiments of transdermal patch disposal devices are disclosed. Each such disposal device utilizes a containment having a patch disposal slot. A transdermal patch may be directed through this patch disposal slot for retention within an internal chamber of the containment. This patch disposal slot may be configured to reduce the potential of being able to withdraw a transdermal patch back out of the containment through the patch disposal slot. The interior of the containment may include one or more ribs which may reduce the potential of being able to withdraw a transdermal patch back out of the containment through the patch disposal slot.

### CROSS-REFERENCE TO RELATED APPLICATIONS

This patent application claims priority to pending U.S. Provisional Patent Application Ser. No. 61/392,547, that is entitled “DISPOSABLE TRANSDERMAL PATCH CONTAINMENT,” and that was filed on Oct. 13, 2010.

### FIELD OF THE INVENTION

The present invention generally relates to the field of transdermal patches and, more particularly, to disposal of transdermal patches.

### BACKGROUND

Abuse, misuse, and overdose of pharmaceutical products (e.g., pain management drugs) are serious health concerns that affect many people on a daily basis all over the world. For instance, diversion and subsequent misuse or abuse may occur when a patient gets a prescription for a pharmaceutical product and does not use all of the pharmaceutical product for whatever reason (e.g., a doctor may prescribe a pharmaceutical product for a patient and advise the patient to take the pharmaceutical product on an “as needed” basis; a patient may be advised to use an entire prescribed amount of pharmaceutical product, but may unilaterally decide to discontinue use of the pharmaceutical product as one or more symptoms disappear). In any case, remaining pharmaceutical product may be ultimately acquired by an individual other than for whom the pharmaceutical product was originally prescribed (e.g., transferred by the original patient to another other individual, such as family member or friend; stolen). While unused pharmaceutical product may be disposed of in the trash, this may not be viewed by some as a secure method of disposal.

In the case of transdermal analgesic patches, a used patch may still retain a significant amount of active ingredient in the patch. A used patch can be very dangerous and can even lead to death for people who have not been prescribed the patch. While some patch manufacturers recommend flushing used patches down the toilet, this practice has raised concerns about drug product entering the water supply. In some states, “take back” programs have been instituted, allowing users to request shipping materials in order to ship used or unused pharmaceutical product (e.g., patches) to a certified disposal company. These programs tend to be costly and require several actions by the patient at multiple times.

### SUMMARY

A first aspect of the present invention is generally directed to transdermal patch packaging or a container that includes a container body, a first compartment, and a second compartment. At least one transdermal patch of a first state or condition is located within the first compartment, while at least one transdermal patch of a second state or condition is located within the second compartment. The first and second states or conditions differ in at least some respect. A transdermal patch within the first compartment hereafter may be referred to as a “first transdermal patch,” while a transdermal patch within the second compartment hereafter may be referred to as a “second transdermal patch.”

A number of feature refinements and additional features are applicable to the first aspect of the present invention. These feature refinements and additional features may be used individually or in any combination. As such, each of the following features that will be discussed may be, but are not required to be, used with any other feature or combination of features of the first aspect of the present invention.

One embodiment has the noted “first condition” of the transdermal patches within the first compartment being that the transdermal patches are new or unused (e.g., not yet having been mounted on or adhered to a patient), with the noted “second condition” of the transdermal patches within the second compartment being that the transdermal patches have been used by a patient (e.g., having been mounted on or adhered to a patient). One embodiment has the noted “first condition” of the transdermal patches within the first compartment being that the transdermal patches are contained within individual primary packaging (e.g., within a sealed pouch, jacket, foil wrapping, or the like), with the noted “second condition” of the transdermal patches within the second compartment being that the transdermal patches are in an exposed state or where the individual transdermal patches have been removed from their associated primary packaging before being disposed within the second compartment. Each of these “exposed” transdermal patches may either have been used by a patient (e.g., having been mounted on or adhered to a patient such that pharmaceutical product was delivered transdermally to the patient) or not (e.g., the second compartment may contain one or more transdermal patches that were removed from their associated primary packaging and disposed in the second compartment before being used by a patient). One embodiment has the noted “first condition” of the transdermal patches within the first compartment being that each of these transdermal patches includes a first amount of pharmaceutical product (e.g., a prescribed dose), with the noted “second condition” of the transdermal patches within the second compartment being that each of these transdermal patches includes a second amount of pharmaceutical product (e.g., something less than that prescribed dose, for instance based upon the transdermal patch having been mounted on or adhered to a patient for a period of time such that at least part of its pharmaceutical product was delivered transdermally to the patient), where the first and second amounts are different.

The first compartment may be sized to accommodate storage of multiple first transdermal patches. Any appropriate number of first transdermal patches may be stored in the first compartment, for instance, a number of first transdermal patches that correspond with a desired “prescription period” and/or a “total prescribed dose.” For instance, a patient's prescription may be for a two-week period, where each first transdermal patch is to be used for 48 hours (i.e., a prescription of this type would include 7 first transdermal patches). Each first transdermal patch within the first compartment may be contained within primary packaging (e.g., a sealed pouch or jacket; foiled packaging; packaging that is torn to gain access to a transdermal patch). Unless otherwise noted herein, all discussion regarding a first transdermal patch within the first compartment can be equally applicable to each first transdermal patch within the first compartment.

The second compartment may be sized to accommodate storage of multiple second transdermal patches. Any appropriate number of second transdermal patches may be stored in the second compartment. In one embodiment, the first and second storage compartments are of the same size. In another embodiment, the first storage compartment may be larger than the second storage compartment. In yet another embodiment, the first storage compartment may be smaller than the second storage compartment. Unless otherwise noted herein, all discussion regarding a second transdermal patch within the second compartment can be equally applicable to each second transdermal patch stored within the second compartment.

The first and second compartments may be characterized as being within the container body in at least some respect. The container body may be formed from any appropriate material or combination of materials, such as cardboard (e.g., formed from one or more pieces of cardboard), paperboard, plastic, and the like. Forming the container body from an appropriate plastic or plastic-like material may be preferred since it could increase the difficulty in accessing second transdermal patches from within the second compartment. The container body may be of any appropriate size, shape, configuration, and/or type. For instance, the container body may be formed from a single piece of flat material utilizing one or more fold lines and/or one or more die cuts to provide a desired configuration (e.g., a square or rectangular box of sorts). In one embodiment, the transdermal patch packaging includes a lid and a hinge between the lid and container body (e.g., where the hinge is defined along a fold line). Opening the lid may provide access to each of the first and second compartments. Closing the lid may at least partially conceal (and thereby encompassing totally concealing) each of the first and second compartments.

The first compartment of the container body may include a first open end. This first open end may be oppositely disposed from a base of the container body. As such, when the base of the container body is disposed on a supporting surface, the container body is oriented in an upright position with the first open end being located remote from the supporting surface. The first open end of the container body is sized to expose (e.g., provide access to) at least some first transdermal patches within the first compartment. In some cases, this first open end may be sized so as to expose all first transdermal patches within the first compartment. A patient may then simultaneously see or count all of the first transdermal patches in the first compartment.

The second compartment of the container body may include a second open end. This second open end may be oppositely disposed from a base of the container body. As such, when the base of the container body is disposed on a supporting surface, the container body is oriented in an upright position with the second open end being located remote from the supporting surface. The second open end of the container body may be sized to expose (e.g., provide access to) at least some of the second transdermal patches within the second compartment. In some cases, this second open end may be sized so as to expose all second transdermal patches within the second compartment. A patient may then simultaneously access all of the second transdermal patches in the second compartment.

One or more additional features may facilitate the disposal of unused transdermal patches. The transdermal patch packaging may include a disposal member within the second compartment, and this disposal member may be used in relation to any of the aspects/embodiments of the present invention identified herein. At least one transdermal patch may be bonded to this disposal member (e.g., with the adhesive side of the transdermal patch facing the disposal member). The disposal member may be of any appropriate size shape, and/or configuration. For instance, the disposal member may be in the form of a card or the like that is disposed into a folded configuration to capture one or more transdermal patches between the folded sections (e.g., the card may be folded over onto itself to dispose at least one transdermal patch therebetween). Such a disposal member may incorporate an appropriate adhesive in any appropriate arrangement to in effect seal one or more transdermal patches within the folded configuration.

The disposal member may be in the form of a sheet or a substrate that includes a plurality of discrete zones or regions, each of which can accommodate a separate transdermal patch. A transdermal patch may be disposed on and bonded to such a disposal member in one of the predefined zones. Although this bond could rely upon remaining adhesive on the transdermal patch, in one embodiment each such zone of the disposal member incorporates adhesive of any appropriate type. This adhesive may be distributed within each zone in any appropriate manner.

In the case where a disposal member itself incorporates adhesive for bonding a transdermal patch thereto, it may be desirable to include a release liner or film. A single release liner or film could be disposed over one or more regions of the disposal member that accommodates a transdermal patch. Another option would be to provide a separate release liner or film for each individual region of the disposal member that accommodates a transdermal patch. Although the disposal member could originally include a plurality of zones or regions for a corresponding number of transdermal patches, the disposal member could be configured so that individual sections thereof could be detached from a remainder of the disposal member, and where each such section accommodates a single used transdermal patch.

The transdermal patch packaging may include what is characterized as a cover that is disposed over the second compartment (e.g., so as to at least partially close or enclose the second compartment; the cover being oppositely disposed from the above-noted container body base). This cover may include one or more apertures. Unless otherwise noted herein, all discussion regarding one of these apertures is applicable to each of these apertures. In one embodiment, a cover aperture is sized and/or configured to reduce the potential that a second transdermal patch can be removed from the second compartment through a cover aperture, to impede the ability of an individual to remove a second transdermal patch from the second compartment through a cover aperture, or both.

The second compartment may be in the form of a single space in which one or more second transdermal patches may be stored or disposed. Each of the above-noted cover apertures may then access a common interior space. However, the second compartment could also be subdivided into a plurality of different interior spaces in any appropriate manner, with each subdivided space being accessed by its own cover aperture or set of cover apertures (e.g., there may be a one-to-one relation between cover apertures and subdivided spaces).

Any cover provided over the second compartment may include a plurality of apertures. There may be a separate aperture for each second transdermal patch to be stored in the second compartment (e.g., a one-to-one relation). Each aperture may be of any appropriate size, shape, and/or configuration. For instance, each aperture may be in the form of an axially or linearly extending slot or slit. A cover aperture may be continually open, or a cover aperture may be defined by rupturing or perforating the cover along a predefined path or section (e.g., a scored segment or score-line previously formed in the cover). In the second instance, forming the cover in this manner may provide a visual indication as to how many second transdermal patches are currently within the second compartment. In one embodiment, the number of scored segments of the cover corresponds with the number of second transdermal patches to be stored in the second compartment.

The second compartment may include one or more flaps or tabs that are intended to reduce the potential that a second transdermal patch may be removed from the second compartment through a cover aperture. At least one flap or tab may be disposed below (e.g., at a lower elevation when the transdermal patch packaging is an upright orientation) and at least partially vertically aligned with one or more cover apertures. Consider the case where the second compartment is at least partially defined by a base (e.g., part of the noted container body base), a perimeter wall (e.g., an annular structure or one that extends a full 360° about a common location whether circular, rectangular, square, or the like; collectively part of a container body sidewall and a divider between the first and second compartments; a completely separate structure from a container body sidewall), and the above-noted cover. In one embodiment, the perimeter wall for the second compartment extends upwardly from the base when the transdermal patch packaging is in an upright orientation, such that the cover and base are disposed on and define opposite ends of the second compartment. One or more flaps or tabs may extend from the perimeter wall both toward, but not to, an opposing portion of the perimeter wall as well as the base. One or more flaps or tabs may extend from an underside of the cover toward, but not to, each of the perimeter wall and the base. Each such flap or tab may be characterized as a cantilever, having a fixed end or portion and an oppositely disposed free end. Each such flap or tab may extend in a downward direction toward the base (and at an angle relative to vertical when the transdermal patch packing is in an upright orientation) proceeding in the direction of its corresponding free end.

Introducing a second transdermal patch into the second compartment through a cover aperture may deflect one or more flaps or tabs in a first direction (e.g., by engagement of the used transdermal patch with such a flap/tab; moving its corresponding free end at least generally in the direction of the base). In the event that the second transdermal patch ends up being positioned below such a flap or tab, the flap or tab may move at least partially back toward its original position, which would reduce the space through which the second transdermal patch would have to be directed through to be pulled back out of the second compartment through a cover aperture. Contact between a second transdermal patch within the second compartment and one or more flaps/tabs may also impede the ability to remove a second transdermal patch from the second compartment through a cover aperture.

Any appropriate number of flaps or tabs may be included in the second compartment, and multiple flaps or tabs may be disposed in any appropriate arrangement. In one embodiment, there are a number of vertically spaced or staggered flaps or tabs in the second compartment (e.g., at least two flaps or tabs may be disposed at different elevations when the above-noted base is positioned on a supporting surface such that the transdermal patch packaging is disposed in an upright orientation). One or more flaps or tabs may be positioned on one side of the perimeter wall, while one or more flaps or tabs may be positioned on an opposite side of the perimeter wall. Flaps or tabs that are positioned on one side of the perimeter wall may be vertically staggered in relation to flaps or tabs that are positioned on an opposite side of the perimeter wall.

The second compartment may also include a plurality of flaps that are disposed at a common elevation when the transdermal patch packing is in an upright orientation. Each flap or tab may be associated with its own aperture, and the various flaps may be at least generally disposed in a common orientation and/or elevation when the transdermal patch packaging is in an upright orientation. A flap or tab on one side of the perimeter wall may also be positioned at a common elevation with a corresponding flap or tab positioned on an opposite side of the perimeter wall (e.g., such flaps or tabs may be spaced a common distance from the above-noted base within the second compartment when the container is in an upright position). Any such pair of flaps may be characterized as being oriented as the mirror image of each other. Any such pair of flaps may also be characterized as converging toward a common location proceeding in a direction of their respective free ends.

One or more flaps or tabs may be separately attached to the perimeter wall of the second compartment in any appropriate manner (e.g., via adhesive), one or more flaps or tabs could be integrally formed with the cover, or both. Each such flap or tab may be formed from any appropriate material or combination of materials, and may be of any appropriate size, shape, and/or configuration. In one embodiment, each such flap or tab is formed from or otherwise includes a first material having a melting temperature that is lower than a melting temperature of the container body. As such, after all second transdermal patches have been positioned in the second compartment, the transdermal patch packaging may be heated (e.g., in a microwave) to a temperature which melts the first material, where the melted first material may then come into contact with (e.g., to at least partially encapsulate) the second transdermal patches (e.g., to reduce the potential that these second transdermal patches may be “re-used”).

The transdermal patch packaging may include a containment that is disposed within the container body, where the second compartment is located within an interior of this containment. One embodiment has this containment being movable relative to the container body (e.g., such that the containment may be repeatedly positioned in and/or withdrawn from the container body). Another embodiment has this containment being fixed relative to the container body. The various features discussed above in relation to the second compartment may thereby equally applicable to this containment.

A first material may be disposed within the second compartment. This first material may have a first melting temperature that is less than a second melting temperature of the container body. Heating the transdermal patch packaging to at least the first melting temperature, but not to the second melting temperature, should cause the first material to transition to a different state (e.g., from a solid to a liquid or at least a “flowable” state) so as to come into contact with the various second transdermal patches within the second compartment. In one embodiment, the first material, after first being heated in the noted manner and thereafter allowed to cool to a suitable temperature, may at least partially encapsulate each of the second transdermal patches within the second compartment.

A second aspect of the present invention is generally directed to a transdermal patch containment that includes a perimeter wall, a base, and an end wall that collectively define an internal compartment. The base and end wall are oppositely disposed, and the end wall includes a plurality of slots. At least one transdermal patch is contained within the internal compartment.

A number of feature refinements and additional features are applicable to the second aspect of the present invention. These feature refinements and additional features may be used individually or in any combination. As such, each of the following features that will be discussed may be, but are not required to be, used with any other feature or combination of features of the second aspect of the present invention.

Each of the features addressed above in relation to the noted containment that may be utilized by the first aspect are equally applicable to the transdermal patch containment of the second aspect. Each of the features discussed above in relation to second compartment utilized by the first aspect are equally applicable to the interior compartment utilized by the second aspect. For instance, the transdermal patch containment of this second aspect may contain one or more flaps or tabs in accordance with the discussion presented above with regard to the first aspect. The discussion presented above on the cover apertures that may be used by the first aspect is also equally applicable to the slots on the end wall for the case of this second aspect. The first material addressed above in relation to the first aspect may also be incorporated into the transdermal patch containment of this second aspect.

A third aspect of the present invention is directed to a transdermal patch disposal device. This patch disposal device includes a containment, an internal chamber or compartment within the containment, a patch disposal slot, and at least one transdermal patch disposed within the internal chamber (e.g., a first transdermal patch). The patch disposal slot is nonlinear (i.e., not in a straight line), extends completely through the containment (e.g., a wall of the containment), and accesses the internal chamber.

A fourth aspect of the present invention is directed to a transdermal patch disposal device. This patch disposal device includes a containment having a recess on its exterior, an internal chamber or compartment within the containment, a patch disposal slot, and at least one transdermal patch disposed within the internal chamber (e.g., a first transdermal patch). The patch disposal slot extends completely through the containment (e.g., a wall of the containment), accesses the internal chamber, and is located within the recess.

A fifth aspect of the present invention is directed to a transdermal patch disposal device. This patch disposal device includes a containment, an internal chamber or compartment within the containment, a patch disposal slot, first and second projections that extend within the internal chamber, and at least one transdermal patch disposed within the internal chamber (e.g., a first transdermal patch). The patch disposal slot extends completely through the containment (e.g., a wall of the containment) and accesses the internal chamber. Furthermore, the patch disposal slot extends between the first and second projections, such that the spacing between the first and second projections collectively define at least part of the patch disposal slot (e.g., a “lower” portion of this slot).

A number of feature refinements and additional features are applicable to each of above-noted third, fourth, and fifth aspects of the present invention. These feature refinements and additional features may be used individually or in any combination in relation to each of the third, fourth, and fifth aspects. Initially, each of the third, fourth, and fifth aspects may be used in relation to the “second compartment” of the first aspect. Each of the third, fourth, and fifth aspects may also be used in combination with the second aspect. Each of the patch disposal slot of the third aspect, the patch disposal slot and recess of the fourth aspect, and the patch disposal slot and first and second projections of the fifth aspect: 1) may be used in conjunction with the second compartment of the first aspect; and 2) may be used in relation to one or more of the plurality of the slots for the transdermal patch containment of the second aspect.

The patch disposal slot may be formed on a first wall of the containment. This first wall may define an upper end of the containment. The patch disposal slot may be of a configuration that is other than linear or linearly/axially extending. The patch disposal slot may be other than linear proceeding along the entirety of a length dimension of the patch disposal slot, with the length dimension of the patch disposal slot being its maximum or largest dimension.

The patch disposal slot may be characterized as including first and second slot ends (e.g., spaced along a length dimension of the patch disposal slot). A first part or length segment of the patch disposal slot (i.e., one portion of the patch disposal slot located somewhere between its first and second slot ends) may be disposed so as to not be collinear with a second part or length segment of the patch disposal slot (i.e., another portion of the patch disposal slot located somewhere between its first and second slot ends).

The patch disposal slot may be of a variety of configurations proceeding along its length dimension (e.g., proceeding from a first slot end to a second slot end). One embodiment has the patch disposal slot being at least generally S-shaped (e.g., exhibiting a sinusoidal curve shape) proceeding along its length dimension (e.g., in a top or plan view; in a view corresponding with looking down into the slot, and thereby into the internal chamber). At least part of the patch disposal slot may be characterized as being curved proceeding along its length dimension (e.g., in a top or plan view; in a view corresponding with looking down into the slot, and thereby into the internal chamber).

The patch disposal slot may include first, second, and third sections or segments disposed along a length dimension of the patch disposal slot (e.g., each of the first, second, and third segments may define a different portion of the length of the patch disposal slot), where the second segment is located between the first and third segments, and where the second segment is not collinear with either of the first and third segments. In one embodiment, each of the first, second, and third segments extend axially or linearly in the length dimension of the patch disposal slot. In one embodiment, the first and third segments are disposed in at least substantially parallel relation to one another, while the second segment connects the adjacent ends of the first and third segments. In one embodiment where the first, second, and third segments each extend axially or linearly in the length dimension of the patch disposal slot, a first included angle exists between the first and second segments of the patch disposal slot and is less than 180°, a second included angle exists between the second and third segments of the patch disposal slot and is less than 180°, and where the first and second included angles are of an at least substantially common magnitude.

The containment may include first and second end walls that are oppositely disposed (e.g., an upper wall and a lower wall, respectively), along with third and fourth end walls that are oppositely disposed (e.g., front and rear walls). The first end wall may include the patch disposal slot, the first segment of the patch disposal slot may be located between the third end wall and the second segment of the patch disposal slot, and the third segment of the patch disposal slot may be located between the fourth end wall and the second segment of the patch disposal slot. A length dimension of the patch disposal slot may be characterized as coinciding with the spacing between the third and fourth end walls. In any case, a reference axis may extend between corresponding portions of the third and fourth end walls. In one embodiment, a length dimension of the first and third segments of the patch disposal slot is at least substantially parallel with this reference axis, while a length dimension of the second segment of the patch disposal slot is non-parallel with this reference axis. In another embodiment, a length dimension of each of the first, second, and third segments of the patch disposal slot is non-parallel with this reference axis.

The patch disposal slot may have a width that is of a first magnitude, and the thickness of the first transdermal patch (as it is directed through the patch disposal slot) is of a second magnitude, where the first magnitude (the width of the patch disposal slot) is only slightly larger than the second magnitude (the thickness of the first transdermal patch as it is directed through the patch disposal slot). In one embodiment, the first magnitude (the width of the patch disposal slot) is no more than 18% larger than the second magnitude (the thickness of the first transdermal patch as it is directed through the patch disposal slot).

The patch disposal slot may be characterized as extending between exterior and interior surfaces of the container. The intersection of the slot with this interior surface may define a pair of edges (e.g., the spacing between the two edges may define a width of the patch disposal slot adjacent to the internal chamber). In one embodiment, each of these edges may be characterized as being in the form of a knife edge. Having the entry into the patch disposal slot (i.e., from the direction of the internal chamber or an interior end of the patch disposal slot) include one or more edges should reduce the potential of being able to withdraw a transdermal patch back out of the internal chamber through the patch disposal slot (e.g., and thereby facilitates disposal of transdermal patches contained with the internal chamber). Incorporating one or more edges at this end of patch disposal slot may provide a “mechanical catch” that may impede removing a transdermal patch out from the internal chamber through the patch disposal slot (e.g., a transdermal patch may become “snagged” on one or more of such edges if someone attempts to withdraw a transdermal patch out of the internal chamber through the patch disposal slot).

The patch disposal slot may be characterized as being located within a recess that may be formed on an exterior surface of the containment. This recess may be characterized as being at least generally V-shaped in a cross-sectional view taken perpendicularly to the length dimension of the slot. The surfaces of the containment that define this recess may “lead” into the patch disposal slot. In one embodiment, the width of the recess is progressively reduced proceeding in the direction of the patch disposal slot. The surfaces of the containment that define the recess may be characterized as converging toward one another proceeding in the direction of the patch disposal slot.

The patch disposal slot may be located between first and second chamfers or chamfered surfaces. The first chamfer may lead to the patch disposal slot and may be positioned on a first side of the patch disposal slot, while the second chamfer may lead to the patch disposal slot and may be positioned on a second side of the patch disposal slot (e.g., where the first and second sides of the patch disposal slot are oppositely disposed). The first and second chamfers may each proceed along an entirety of a length of the patch disposal slot. The first and second chamfers may collectively define the above-noted recess.

The first and second chamfers may each be characterized as angled surfaces. In one embodiment, the first and second chamfers are disposed in different orientations. The first chamfer may be the mirror image of the second chamfer. In one embodiment, the first and second chamfers are planar surfaces. Other contours may be appropriate.

The first and second chamfers may each be characterized as surfaces that extend from a common first wall section of the containment to an exterior end of the patch disposal slot. Consider the case where the containment includes first and second slot sidewalls, the spacing between which defines at least part of the width of the patch disposal slot. The first chamfer may extend between the first wall section and the first slot sidewall, and may be disposed in a different orientation than each of the first wall section and the first slot sidewall. Similarly, the second chamfer may extend between the first wall section and the second slot sidewall, and may be disposed in a different orientation than each of the first wall section and the second slot sidewall.

The first and second chamfers may facilitate directing the transdermal patches through the patch disposal slot. For instance, the first and second chamfers may help in directing the first transdermal patch through the patch disposal slot when the width of the patch disposal slot is at most only slightly larger than the thickness of the first transdermal patch as it is being directed through the patch disposal slot. The first and second chamfers may help change the configuration of a transdermal patch so as to at least generally match the configuration of the patch disposal slot (e.g., its length dimension configuration) and/or may otherwise facilitate directing a transdermal patch through a non-linear patch disposal slot for storage in the internal chamber.

First and second projections may extend within the internal chamber (e.g., from an interior surface of the containment, such that the first and second projections may be characterized as cantilevers). The patch disposal slot may extend between these first and second projections such that the first and second projections collectively define at least part of the patch disposal slot (e.g., a lower portion thereof, where “lower” is in the direction of passing a transdermal patch through the patch disposal slot for storage within the internal chamber). A pair of spaced slot sidewalls may define the patch disposal slot. Part of each slot sidewall may be defined by a wall thickness of the containment (e.g., an upper portion of the patch disposal slot). Part of each slot sidewall may be defined by a sidewall of the first or second projection. In one embodiment: an intersection between a sidewall of the first projection and an end wall of the first projection define an edge (e.g., a knife edge) at the adjacent end of the patch disposal slot (e.g., an interior end of the patent disposal slot); and an intersection between a sidewall of the second projection and an end wall of the second projection define an edge (e.g., a knife edge) at the adjacent end of the patch disposal slot (e.g., an interior end of the patent disposal slot).

The configuration of the patch disposal slot may reduce the potential that a transdermal patch can be removed from the internal chamber of the containment. The interior of the containment may include one or more features that also address reducing the potential of being able to withdraw a transdermal patch from the internal chamber of the containment. One or more ribs may be positioned within the internal chamber of the containment. In one embodiment, multiple ribs extend from an interior surface of each of a pair of opposing walls of the containment (e.g., first and second sidewalls, noted below). Each rib may be characterized as extending from an interior surface of the containment in the direction of a projection of the patch disposal slot into the internal chamber. Utilizing one or more internal ribs may obstruct the withdrawal of a transdermal patch from the internal chamber of the containment. Any such internal rib(s) may also provide structural reinforcement for the containment.

The containment may include first and second end walls that are oppositely disposed (e.g., an upper wall and a lower wall, respectively), third and fourth end walls that are oppositely disposed (e.g., front and rear walls), and first and second sidewalls that are oppositely disposed. The first sidewall may intersect with each of the first, second, third, and fourth end walls. Similarly, the second sidewall may intersect with each of the first, second, third, and fourth end walls. The first end wall may include the patch disposal slot, a length dimension of the patch disposal slot may coincide with a spacing between the third and fourth end walls, or both. At least part of each of the first and second sidewalls may be convex proceeding in a dimension coinciding with the spacing between the first and second end walls (e.g., upper and lower walls), at least part of the third and fourth end walls (e.g., front and rear walls) may be concave proceeding in a dimension coinciding with the spacing between the first and second end walls (e.g., upper and lower walls), or both. In one embodiment, at least part of each of the third and fourth end walls (e.g., front and rear walls) may also be convex proceeding in a dimension coinciding with the spacing between the first and second sidewalls.

Consider the case where the above-noted first and second end walls define upper and lower walls, respectively, of the containment, and where the above-noted third and fourth end walls define front and rear walls, respectively, of the containment. In this case, at least part of the first and second sidewalls may be at least generally curved or bowed outwardly relative to the interior of the containment, at least part of the front and rear walls of the containment may be at least generally curved or bowed inwardly relative to the interior of the containment in a first dimension (e.g., the dimension coinciding with the spacing between the upper and lower walls), or both. At least part of each of the third and fourth end walls (e.g., front and rear walls) may also be curved or bowed outwardly relative to the interior of the containment in a second dimension (e.g., the dimension coinciding with the spacing between the noted first and second sidewalls). In one embodiment, the third and fourth end walls each utilize a saddle-like configuration.

A sixth aspect of the present invention is directed to a disposal method for transdermal patches that utilizes a containment. This containment includes an internal chamber and a patch disposal slot. A first transdermal patch is directed through the patch disposal slot to gain access to the internal chamber. The first transdermal patch may be changed (e.g., bent or twisted) from a first configuration into a second configuration, for instance so as to at least generally conform with a shape/dimension of the patch disposal slot (e.g., a patch disposal slot of the type addressed by the third aspect). This configurational change of the first transdermal patch may be used in relation to directing the first transdermal patch through the patch disposal slot. The first transdermal patch is biased from the second configuration back toward its first configuration once the first transdermal patch is within the internal chamber.

A number of feature refinements and additional features are applicable to the sixth aspect of the present invention. These feature refinements and additional features may be used individually or in any combination. As such, each of the following features that will be discussed may be, but are not required to be, used with any other feature or combination of features of the sixth aspect of the present invention. Initially, the transdermal patch disposal device of the third aspect may be utilized in the disposal method of this sixth aspect.

The first transdermal patch may be folded prior to being directed through the patch disposal slot (e.g., folded in half). The first transdermal patch may be retained in this folded configuration, for instance via remaining adhesive on a surface of the first transdermal patch that is used to adhere the transdermal patch to a patient.

Consider the case where a perimeter of the first transdermal patch is defined by four edges in preparation for directing the first transdermal patch through the patch disposal slot (e.g., after being folded). First and second edges of the first transdermal patch may be disposed in opposing relation, and third and fourth edges of the first transdermal patch may also be disposed in opposing relation. The first and second edges of the first transdermal patch thereby may define opposing first and second ends of the first transdermal patch. The first transdermal patch may incorporate one or more bends between these opposing first and second ends.

The configurational change of the first transdermal patch may be characterized as elastically deforming the first transdermal patch (e.g., such that the elasticity of the first transdermal patch will move the first transdermal patch from its second configuration at least back toward its first configuration after having passed through the patch disposal slot and gained access to the internal chamber, including where the elasticity of the first transdermal patch is the only force used for this change of configuration). The configurational change of the first transdermal patch may be characterized as storing energy in the first transdermal patch, and this energy may be used to move the first transdermal patch from its second configuration at least back toward its first configuration after having passed through the patch disposal slot and gained access to the internal chamber (e.g., including where this stored internal energy is the only force used for this change of configuration).

The first transdermal patch may be characterized as moving from its second configuration at least back toward its first configuration once it gains access to the internal chamber (e.g., by biasing forces possessed by the first transdermal patch). The first transdermal patch could return all the way back to its first configuration, or the first transdermal patch could return to an intermediate configuration between the first and second configurations (e.g., a third configuration). In any case, changing the first transdermal patch from its second configuration (once within the internal chamber) should produce a configuration for the first transdermal patch that no longer matches a configuration of the patch disposal slot. This should reduce the potential of being able to withdraw a transdermal patch back out of the internal chamber through the patch disposal slot (e.g., and thereby facilitates disposal of transdermal patches contained with the internal chamber).

Various embodiments are described herein that utilize a slot through which a transdermal patch is directed for purposes of and/or in preparation for disposal of the corresponding containment. It may be desirable to place tape over each such slot prior to disposing of the containment (e.g., prior to placing the containment in the trash). One or more pieces of tape could be utilized. Appropriate information could be printed on any such tape, for instance information for purposes of complying with regulatory requirements (e.g., identifying the name of the active ingredient(s) associated with the transdermal patches).

Each transdermal patch utilized with the present invention may include any appropriate pharmaceutical product. Examples of appropriate pharmaceutical products that may be included in such transdermal patches include (but are not limited to): U.S. Drug Enforcement Administration (DEA) scheduled (e.g., Schedule II) drugs such as fentanyl, lidocaine, tetracaine, prilocaine, thebaine, buprenorphine, sufentanil, alfentanil, codeine, dihydrocodeine, hydrocodone, hydromorphone, levorphanol, methadone, morphine, nalbuphine, noscapine, opium, oxycodone, and propoxyphene; non-steroidal anti-inflammatory drugs (NSAIDs) such as ketoprofen, diclofenac, flurbiprofen, and ibuprofen; steroids such as testosterone and estradiol; psychoactive drugs such as buspirone; vitamins such as vitamin B12; vasodilators such as nitroglycerin; vaccines; antiemetics; capsaicin; and nicotine. Further, any transdermal patches utilized with the present invention can function to provide drug delivery in any appropriate manner. For instance, such transdermal patches may include those functioning via a passive delivery mechanism (e.g., pharmaceutical product located within the adhesive of the patch, within a reservoir of the patch, within a semisolid matrix (e.g., a gel)) or via an active delivery mechanism (e.g., iontophoresis, sonophoresis, electroporation, microneedles, abrasion, needle-less injection, suction, stretching, magnetophoresis, radio frequency, lasers, photomechanical waves, temperature (e.g., heat-activation)).

Any feature of any other various aspects of the present invention that is intended to be limited to a “singular” context or the like will be clearly set forth herein by terms such as “only,” “single,” “limited to,” or the like. Merely introducing a feature in accordance with commonly accepted antecedent basis practice does not limit the corresponding feature to the singular (e.g., indicating that a compartment includes “a transdermal patch” alone does not mean that the compartment includes only a single transdermal patch). Moreover, any failure to use phrases such as “at least one” also does not limit the corresponding feature to the singular (e.g., indicating that a compartment includes “a transdermal patch” alone does not mean that the compartment includes only a single transdermal patch). Use of the phrase “at least generally” or the like in relation to a particular feature encompasses the corresponding characteristic and insubstantial variations thereof (e.g., indicating that a container body is at least generally rectangular encompasses the container body being rectangular). Finally, a reference of a feature in conjunction with the phrase “in one embodiment” does not limit the use of the feature to a single embodiment.

### BRIEF DESCRIPTION OF THE FIGURES

FIG. 1 is a perspective view of one embodiment of transdermal patch packaging.

FIG. 2 is a perspective view of the transdermal patch packaging of FIG. 1, with an open lid to illustrate its multiple internal storage compartments for transdermal patches.

FIG. 2A is a schematic of a transdermal patch within primary packaging.

FIG. 3 is a perspective view of another embodiment of transdermal patch packaging with multiple internal storage compartments for transdermal patches.

FIG. 4A is a perspective view of one embodiment of a separate containment for transdermal patches.

FIG. 4B is a perspective view of a portion of two of the slots used by the containment of FIG. 4A to access an interior compartment in which transdermal patches may be disposed, and further illustrating a corresponding flap that may be utilized.

FIG. 4C is a cross-sectional view of the containment of FIG. 4A taken along line C-C.

FIG. 5 is a cross-sectional view of another embodiment of a containment with slots for accessing an internal compartment for storage of transdermal patches, and that have a corresponding flap.

FIG. 6 is a cross-sectional view of another embodiment of a containment with slots for accessing an internal compartment for storage of transdermal patches, and which incorporates a plurality of vertically-spaced flaps.

FIG. 7A is a perspective view of a transdermal patch containment with a heat-activated encapsulating material.

FIG. 7B is a cross-sectional view of the containment of FIG. 7A before being heated to a melting temperature of the encapsulating material.

FIG. 7C is a cross-sectional view of the container of FIG. 7A after being heated to at least the melting temperature of the encapsulating material.

FIG. 8 is an embodiment of a transdermal patch disposal member in the form of a sheet, and that may be used in conjunction with other secondary packaging for the disposal of transdermal patches.

FIG. 9 is an embodiment of a transdermal patch disposal member in the form of a foldable disposal card, and that may be used in conjunction with other secondary packaging for the disposal of one or more transdermal patches.

FIG. 10 is a perspective view of one embodiment of a transdermal patch disposal device that utilizes a non-linear patch disposal slot.

FIG. 11 is a cross-sectional view that illustrates the patch disposal slot from the transdermal patch disposal device of FIG. 10.

FIG. 12A is an edge view of a folded transdermal patch in a first configuration.

FIG. 12B is an edge view of a folded transdermal patch in a second configuration for directing the same through the patch disposal slot utilized by the transdermal patch disposal device of FIG. 10.

FIG. 13 is a cross-sectional view that illustrates a pair of projections that may be used in conjunction with a patch disposal slot of a transdermal patch disposal device.

FIG. 14 is a perspective view of one embodiment of a transdermal patch disposal device that utilizes a patch disposal slot located within a recess.

FIG. 15 is a cross-sectional view that illustrates the patch disposal slot from the transdermal patch disposal device of FIG. 14.

FIG. 16A is a top or plan view of one embodiment of a transdermal patch disposal device having a non-linear patch disposal slot located within a recess.

FIG. 16B is a perspective view of the transdermal patch disposal device of FIG. 16A.

FIG. 16C is a plan view of the patch disposal slot used by the transdermal patch disposal device of FIG. 16A.

FIG. 17A is a perspective view of another embodiment of a transdermal patch disposal device that utilizes a non-linear patch disposal slot, along with internal patch retention ribs.

FIG. 17B is a side view of the transdermal patch disposal device of FIG. 17A.

FIG. 17C is a front view of the transdermal patch disposal device of FIG. 17A.

FIG. 17D is a cutaway view of the transdermal patch disposal device of FIG. 17A, taken along line D-D in FIG. 17B.

FIG. 17E is a plan view of the patch disposal slot used by the transdermal patch disposal device of FIG. 17A.

FIG. 17F is a perspective view of part of the transdermal patch disposal device of FIG. 17A, illustrating a plurality of internal patch retention ribs.

### DETAILED DESCRIPTION

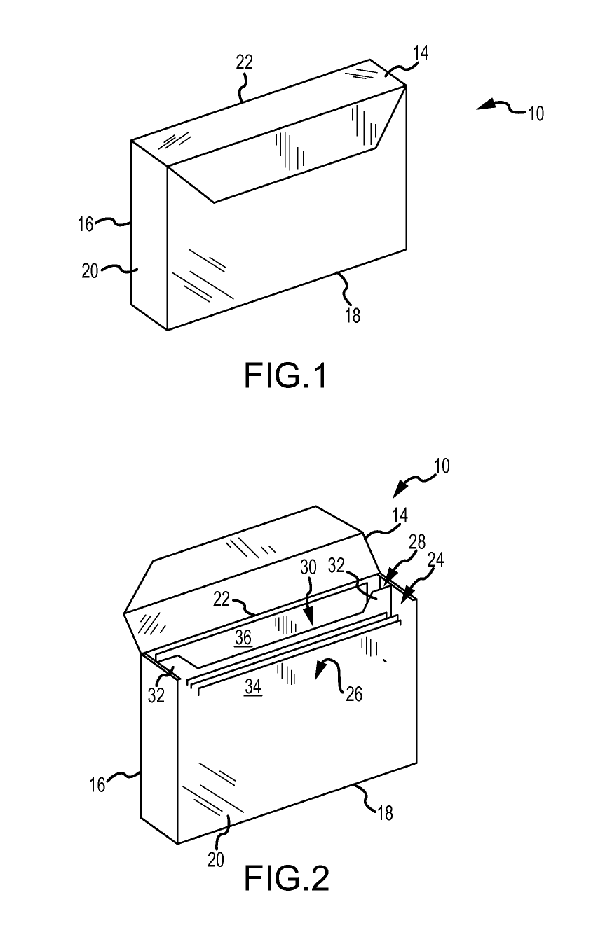

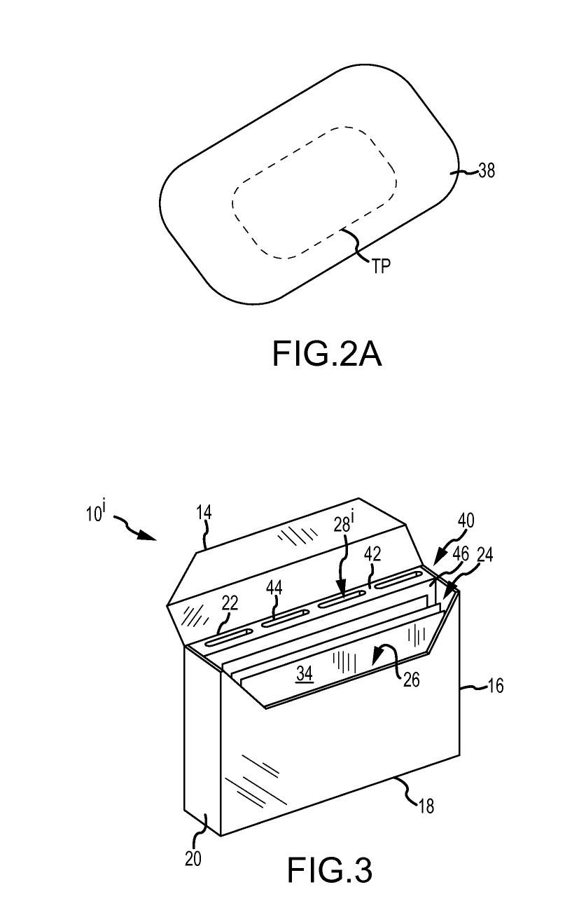

FIGS. 1 and 2 illustrate one embodiment of a transdermal patch container or transdermal patch packaging 10. The transdermal patch container 10 includes a container body 16 and a lid 14. The lid 14 is movable relative to the container body 16 by a hinge 22 of any appropriate type (e.g., a fold line between the lid 14 and container body 16). Generally, the lid 14 may be disposed in the closed position of FIG. 1, and may be moved relative to the container body 16 to the open position shown in FIG. 2 to provide access to the interior of the transdermal patch container 10. Other types of lids could be used the transdermal patch container 10, for instance lids that are totally removable from the container body 16 (e.g., via a detachable interconnection (e.g., snap lock, threads) such that the lid could be attached, removed, and reattached to the container body 16 without damaging either the lid and/or container body 16).

The container body 16 includes a base 18 and a container body sidewall 20. The base 18 is disposed opposite of the above-noted lid 14. Disposing the base 18 on an appropriate supporting surface in turn disposes the transdermal patch container 10 in an upright position. The lid 14 and container body 16 may be of any appropriate size, shape, and/or configuration. In one embodiment, the lid 14 and container body 16 are integrally formed (e.g., a unitary structure without any joint(s) between the lid14 and container body 16). The lid 14 and container body 16 each may be formed from any appropriate material or combination of materials. For instance, the lid 14 and container 16 may be formed from cardboard, paperboard, plastic, or the like.

The transdermal patch container 10 includes a plurality of separate storage areas. FIG. 2 shows the lid 14 in an open position that exposes a first compartment 24 and a second compartment 28. A divider 32 separates the first compartment 24 from the second compartment 28, for instance by extending between opposing portions of the container body sidewall 20. As such, the first compartment 24 is defined by a first portion of the container body sidewall 20 and the divider 32, while the second compartment 28 is defined by a second portion of the container body sidewall 20 and the divider 32.

Access to each of the first compartment 24 and the second compartment 28 is controlled by the lid 14 of the transdermal patch container 10. Disposing the lid 14 in its open position (e.g., FIG. 2) exposes a first opening 26 that provides access to an entirety of the first compartment 24. Disposing the lid 14 in its open position (e.g., FIG. 2) also simultaneously exposes a second opening 30 that provides access to an entirety of the second compartment 28.

One or more first transdermal patches 34 may be stored in the first compartment 24, while one or more second transdermal patches 36 may be simultaneously stored in the second compartment 28. The divider 32 at least somewhat isolates (e.g., physically) the first compartment 24 from the second compartment 28. Therefore, the first transdermal patches 34 should in turn be physically isolated from the second transdermal patches 36. The first compartment 24 may be sized to accommodate any appropriate number of first transdermal patches 34. Similarly, the second compartment 28 may be sized to accommodate any appropriate number of second transdermal patches 36.

The first transdermal patches 34 may be individually contained within appropriate primary packaging (e.g., within a sealed pouch, jacket, foil wrapping, or the like). FIG. 2A shows a transdermal patch TP within representative primary packaging 38 of this type. The transdermal patch TP may be of any appropriate shape and of any appropriate configuration. The transdermal patch TP may include any appropriate pharmaceutical product. Examples of appropriate pharmaceutical products that may be included in such transdermal patches include (but are not limited to): U.S. Drug Enforcement Administration (DEA) scheduled (e.g., Schedule II) drugs such as fentanyl, lidocaine, tetracaine, prilocaine, thebaine, buprenorphine, sufentanil, alfentanil, codeine, dihydrocodeine, hydrocodone, hydromorphone, levorphanol, methadone, morphine, nalbuphine, noscapine, opium, oxycodone, and propoxyphene; non-steroidal anti-inflammatory drugs (NSAIDs) such as ketoprofen, diclofenac, flurbiprofen, and ibuprofen; steroids such as testosterone and estradiol; psychoactive drugs such as buspirone; vitamins such as vitamin B12; vasodilators such as nitroglycerin; vaccines; antiemetics; capsaicin; and nicotine. Further, the transdermal patch TP can function to provide drug delivery in any appropriate manner. For instance, the transdermal patch TP may include those functioning via a passive delivery mechanism (e.g., pharmaceutical product located within the adhesive of the patch, within a reservoir of the patch, within a semisolid matrix (e.g., a gel)) or via an active delivery mechanism (e.g., iontophoresis, sonophoresis, electroporation, microneedles, abrasion, needle-less injection, suction, stretching, magnetophoresis, radio frequency, lasers, photomechanical waves, temperature (e.g., heat-activation)).

The first transdermal patches 34 may differ from the second transdermal patches 36 in one or more respects. The first transdermal patches 34 may be characterized as being new or unused (e.g., not yet having been mounted on or adhered to a patient), while the second transdermal patches 36 may be characterized as having been used by a patient (e.g., having been mounted on or adhered to a patient). The first transdermal patches 34 may be individually contained within appropriate primary packaging, while the second transdermal patches 36 may be in an exposed state or where the individual second transdermal patches 36 have been removed from their associated primary packaging before being disposed within the second compartment 28. Each of the “exposed” second transdermal patches 36 may either have been used by a patient (e.g., having been mounted on or adhered to a patient such that pharmaceutical product was delivered to the patient) or not (e.g., the second compartment 28 may contain one or more transdermal patches that were removed from their associated primary packaging and disposed in the second compartment 28 before being used by a patient). The first transdermal patches 34 may include a first amount of pharmaceutical product (e.g., a prescribed dose), while the second transdermal patches 36 may include a second amount of pharmaceutical product (e.g., something less than the prescribed dose, for instance based upon the transdermal patch having been mounted on or adhered to a patient for a period of time such that at least part of its pharmaceutical product was delivered to the patient), where the first and second amounts are different.

In one embodiment, the transdermal patch container 10 is prescribed to a patient with a predetermined number of first transdermal patches 34 within the first compartment 24. As the patient goes through the prescription, the patient may dispose of the second transdermal patches 36 by placing the same in the second compartment 28. Once the patient has used the entire prescription, all of the first transdermal patches 34 that were originally provided should now be within the second compartment 28 in the form of second transdermal patches 36 (e.g., there would no longer be any first transdermal patches 34 within the first compartment 24 in this instance).

A variation of the transdermal patch container 10 of FIGS. 1-2 is presented in FIG. 3 and is identified by reference numeral 10ⁱ. Corresponding components between the embodiments of FIG. 3 and FIGS. 1-2 are identified by a common reference numeral. Those corresponding components that differ in at least some respect are identified by a superscripted “i” designation in FIG. 3.

The transdermal patch container 10ⁱ includes a containment 40 having an enclosed second compartment 28ⁱ for storage of second transdermal patches 36. This containment 40 could be removably disposed within the container body 16 of the transdermal patch container 10ⁱ (e.g. such that the containment 40 could be removed from and inserted into the container body 16; in the form of an autonomous structure). Alternatively, the containment 40 could be fixed relative to the container body 16 in any appropriate manner (e.g., via one or more adhesives). In any case, the containment 40 includes an upper end wall or cover 42 that is disposed opposite of the base 18 of the container body 16. The containment 40 also includes a perimeter wall 46 that extends a full 360° about the perimeter of the second compartment 28ⁱ. The perimeter wall 46 could be defined by one or more of the above-noted divider 32 and the above-noted second portion of the container body sidewall 20. The perimeter wall 46 could also be a completely separate structure from each of the divider 32 and the above-noted second portion of the container body sidewall 20. In one embodiment, the divider 32 shown in the embodiment of FIGS. 1-2 could be integrally formed with the upper end wall 42 shown in FIG. 3 and is used to define an enclosed space for the containment 40 (along with the above-noted second portion of the container body sidewall 20).

The noted upper end wall 42 includes one or more apertures or slots 44 to provide access to the enclosed second compartment 28ⁱ of the containment 40. Each such slot 44 may be defined in any appropriate manner, and furthermore may be of any appropriate size, shape, and/or configuration. Any appropriate number of slots 44 may be utilized. Multiple slots 44 may be disposed in any appropriate arrangement. In one embodiment, the slots 44 are in the form of pre-existing structures. In another embodiment, the slots 44 may be defined by a user perforating the containment upper end wall 42 along a predetermined path (e.g., along a scored segment). This may also be utilized to provide an indication of how many second transdermal patches 36 have been disposed in the second compartment 28ⁱ (e.g., instructions may be provided for users to only insert one second transdermal patch 36 through any one slot 44). Each of the slots 44 could also include any appropriate labeling (e.g., “used patch #1”, “used patch #2”, etc, or the like).

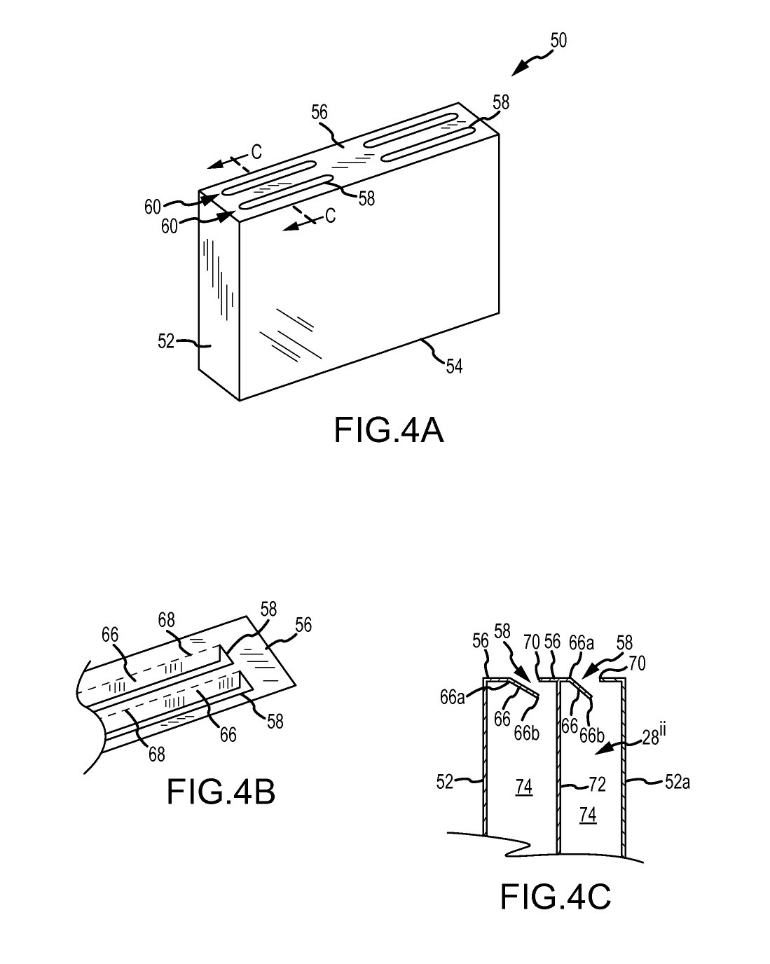

One embodiment of a separate containment for second transdermal patches 36 is illustrated in FIGS. 4A-C and is identified by reference numeral 50. Generally, the containment 50 could be positioned within the second compartment 28 of the transdermal patch container 10 of FIGS. 1-2. Another option would be for the transdermal patch container 10 of FIGS. 1-2 to eliminate the above-described divider 32, and to instead use the containment 50 of FIGS. 4A-C to provide physical separation between any second transdermal patches 36 in the containment 50 and the first transdermal patches 34 in its first compartment 24.

The containment 50 of FIG. 4A includes a perimeter wall 52, a base 54 (e.g., a portion of the container body base 18), and an upper end wall 56. When the containment base 54 is disposed on an appropriate supporting surface, the containment 50 is disposed in an upright orientation. Like the containment 40 of FIG. 3, the upper end wall 56 includes one or more apertures or slots 58. A single row of slots 44 is shown for the FIG. 3 embodiment, while two rows 60 of slots 58 are shown for the embodiment of FIGS. 4A-C. Each of the containments 40, 50 may utilize any appropriate number of slot rows, and any appropriate number of slots may provided for each slot row. The discussion presented above with regard to the slots 44 used by the embodiment of FIG. 3 is equally applicable to the slots 58 used by the embodiment of FIG. 4A, unless otherwise specifically noted to the contrary.

Generally, the perimeter wall 52, base 54, and upper end wall 56 collectively define an enclosed space in the form of a second compartment 28ⁱⁱ for receiving second transdermal patches 36. FIG. 4C illustrates that the second compartment 28ⁱⁱ is actually subdivided into two separate sub-compartments 74 by an internal divider 72. The second compartment 28ⁱⁱ could be divided up into any appropriate number of sub-compartments 74 and in any appropriate manner. The second compartment 28ⁱⁱ could also be in the form of a single continuous space (e.g., each slot 58 could access the same common interior space—not shown).

FIGS. 4B and 4C illustrate that the containment 50 utilizes a separate flap or tab 66 in conjunction with each slot 58 (e.g., each slot 58 may have its own dedicated flap 66). Such flaps or tabs 66 could be used by the embodiment of FIG. 3 as well. In any case, a flap 66 may be utilized to reduce the potential that a second transdermal patch 36 can be withdrawn out of the second compartment 28ⁱⁱ (or any other internal compartment disclosed herein) through its corresponding slot 58. In the illustrated embodiment, the various flaps 66 are integrally formed with the upper end wall 56. For instance and as discussed above in relation to the FIG. 3 embodiment, the slots 58 may be defined by a user perforating the upper end wall 56 along a predetermined path (e.g., along a scored segment 70—where a flap 66 separates from a remainder of the upper end wall 56). The part of each flap 66 that remains attached or connected to the containment upper end wall 56 may include a hinge 68 (e.g., a fold or fold line) about which the flap 66 may move. A flap 66 could also be separately attached to the containment upper end wall 56 (not shown). In any case, each flap 66 may be characterized as a cantilever—having a fixed end 66a (where it extends from the upper end wall 56) and an oppositely disposed free end 66b.

FIG. 4C illustrates that each flap 66 is disposed at an acute angle relative to horizontal, and furthermore extends from the upper end wall 56 both at least generally in the direction of the containment base 54 (e.g., extending in a downward proceeding away from the upper end wall 56 and toward its free end 66b when the containment 50 is in an upright position) and at least generally in the direction of a common perimeter wall section 52a (e.g., the flaps 66 may be disposed in an at least generally common orientation, and including being disposed in parallel relation). Each flap 66 may be characterized as being positioned at least partially below its corresponding slot 58. As such, directing a second transdermal patch 36 into a slot 58 may deflect the corresponding flap 66 in a downward direction (e.g., by moving its free end 66b in at least generally in the direction of the base 54 and at least generally about its corresponding hinge 68). If the second transdermal patch 36 is directed completely past this flap 66, the flap 66 could move at least partially back toward its original position, which should reduce the potential that a second transdermal patch 36 could be withdrawn back out of the second compartment 28ⁱⁱ through its corresponding slot 58. Even if a second transdermal patch 36 is not directed completely past the flap 66, pulling back up on such second transdermal patch 36 may cause the free end 66b of the corresponding flap 66 to move at least generally upwardly in the direction of the upper end wall 56 and at least generally about its corresponding hinge 68, and which may restrain this upwardly-directed movement of the second transdermal patch 36.

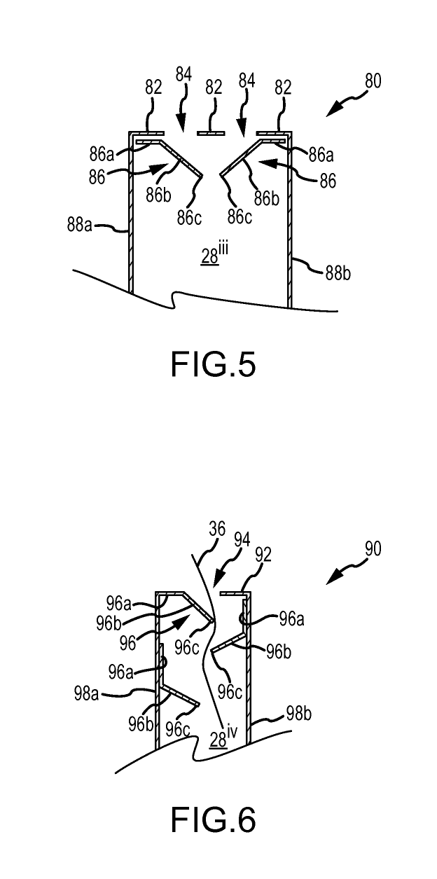

Another embodiment of a containment for second transdermal patches 36 is illustrated in FIG. 5 and is identified by reference numeral 80. Generally, the containment 80 could be an autonomous structure and positioned within the second compartment 28 of the transdermal patch container 10 of FIGS. 1-2. Another option would be for the transdermal patch container 10 of FIGS. 1-2 to eliminate the above-described divider 32, and to instead use the containment 80 to provide physical separation between any second transdermal patches 36 in the containment 80 and any first transdermal patches 34 in its first compartment 24. Yet another option would be for the containment 80 of FIG. 5 to be used in place of the containment 40 of FIG. 3 as described above.

The containment 80 of FIG. 5 includes a perimeter wall that includes a first perimeter wall section 88a and a second perimeter wall section 88b, a base (not shown), and an oppositely disposed upper end wall 82. When the base of the containment 80 is disposed on an appropriate supporting surface, the containment 80 will be disposed in an upright orientation. Like the containment 50 of FIGS. 4A-C, the upper end wall 82 of the containment 80 includes a plurality of apertures or slots 84. More specifically, the containment 80 includes two rows of slots 84 (e.g., similar to the rows 60 shown in FIG. 4A), with each such row having any appropriate number of slots 84. The discussion presented above with regard to the slots 44 used by the embodiment of FIG. 3 again is equally applicable to the slots 84 used by the embodiment of FIG. 5.

Generally, the perimeter wall, base, and upper end wall 82 of the containment 80 collectively define an enclosed space in the form of a second compartment 28ⁱⁱⁱ for receiving second transdermal patches 36. The second compartment 28ⁱⁱⁱ is in the form of a single continuous space in the illustrated embodiment (e.g., each of the slots 84 access the same, common interior space). The second compartment 28ⁱⁱⁱ could also be subdivided into multiple sub-compartments in the manner discussed above in relation to the embodiment of FIGS. 4A-C, where two or more adjacent rows of slots 84 would each access a common sub-compartment (not shown).

FIG. 5 illustrates that the containment 80 may utilize a separate flap or tab 86 in conjunction with each slot 84 (e.g., each slot 84 may have its own dedicated flap 86). Each flap 86 could be incorporated by the containment 80 in the manner discussed above with regard to the containment 50 of FIGS. 4A-C (e.g., where the various flaps 86 could be characterized as being integrally formed with the upper end wall 82). However and in the illustrated embodiment of FIG. 5, each flap 86 is actually a separate structure from the upper end wall 82, and includes a first flap section 86a that is appropriately secured to the underside of the upper end wall 82 (e.g., via an adhesive), as well as a second flap section 86b that extends at least generally in the direction of the base 54 (e.g., extends downwardly from the upper end wall 82 when the containment 80 is in an upright position) and at least generally in the direction of the second flap section 86b of an adjacent flap 86. The orientation of the flap(s) 86 in one row of slots 84 may be at least generally the mirror image of the flap(s) 86 in an adjacent row of slots 84. The flap(s) 86 in one row of slots 84 and the flap(s) 86 in an adjacent row of slots 84 may also be characterized as at least generally converging toward each other progressing in the direction of their respective free ends 86c. Yet another characterization is that the containment 80 includes at least one pair of adjacently disposed slots 84, where the flap 86 for one slot 84 of this pair extends at least generally in the direction of the base and also least generally in the direction of the above-noted second perimeter wall section 88b, and where the flap 86 for the other slot 84 of this pair extends at least generally in the direction of the base and also least generally in the direction of the above-noted first perimeter wall section 88a.

As in the case of the embodiment of FIGS. 4A-C, each flap 86 may be characterized as a cantilever—having a fixed end (e.g., its first flap section 86a) and an oppositely disposed free end 86c. Each flap 86 may be characterized as being positioned at least partially below its corresponding slot 84. The flaps 86 of the containment 80 of FIG. 5 may function in the manner discussed above with regard to the flaps 66 of the containment 50 of FIGS. 4A-C with regard to at least reducing the potential that second transdermal patches 36 can be removed from the second compartment 28ⁱⁱⁱ through one or more of the slots 84.

Another embodiment of a containment for second transdermal patches 36 is illustrated in FIG. 6 and is identified by reference numeral 90. Generally, the containment 90 could be an autonomous structure and positioned within the second compartment 28 of the transdermal patch container 10 of FIGS. 1-2. Another option would be for the transdermal patch container 10 of FIGS. 1-2 to eliminate the above-described divider 32, and to instead use the containment 90 to provide separation between any second transdermal patches 36 in the containment 90 and first transdermal patches 34 in its first compartment 24. Yet another option would be for the containment 90 of FIG. 6 to be used in place of the containment 40 of FIG. 3 as described above.

The containment 90 of FIG. 6 includes a perimeter wall having a first perimeter wall section 98a and a second perimeter wall section 98b, a base (not shown; e.g., part of the container body base 18), and an oppositely disposed upper end wall 92. When its base is disposed on an appropriate supporting surface, the containment 90 is disposed in an upright orientation. In any case, the upper end wall 92 includes one or more slots or apertures 94. The discussion presented above with regard to the slots 44 used by the embodiment of FIG. 3 is equally applicable to the slots 94 used by the embodiment of FIG. 6.

Generally, the perimeter wall, base, and upper end wall 92 of the containment 90 collectively define an enclosed space in the form of a second compartment 28^{iv} for receiving second transdermal patches 36. The second compartment 28^{iv} is in the form of a single continuous space in the illustrated embodiment. The second compartment 28^{iv} could also be subdivided into multiple sub-compartments in the manner discussed above in relation to the embodiment of FIGS. 4A-C.

FIG. 6 illustrates that the containment 90 may utilize a plurality of flaps or tabs 96 that are disposed in vertically spaced or staggered relation when the containment 90 is in the illustrated upright position or orientation. In one embodiment, the length dimension of each of the flaps 96 shown in FIG. 6 coincides with the length of at least one slot 96. In the case where there are a plurality of slots 94 disposed in spaced relation and in a common row, each of the flaps 96 shown in FIG. 6 could extend along the entire length or any portion of the length of such a row.

The uppermost flap 96 shown in FIG. 6 is positioned at least partially below at least one slot 94, and may be incorporated by the containment 90 in any appropriate manner. In the illustrated embodiment, the uppermost flap 96 includes a first flap section 96a that is appropriately attached to an underside of the upper end wall 92. A second flap section 96b of this uppermost flap 96 extends downwardly and also toward the above-noted second perimeter wall section 98b of the containment 90. The next flap 96 (proceeding downwardly in the vertical dimension when the containment 90 is upright) has its first flap section 96a appropriately attached to the second perimeter wall section 98b of the containment sidewall 98, and its corresponding second flap section 96b extends downwardly and toward the oppositely disposed first perimeter wall section 98a of the containment 90. The next flap 96 (again proceeding downwardly in the vertical dimension when the containment 90 is upright) has its first flap section 96a appropriately attached to the first perimeter wall section 98a of the containment 90, and its corresponding second flap section 96b extends downwardly and also toward the oppositely disposed second perimeter wall section 98b of the containment 90.

The flaps 96 that extend toward a common perimeter wall section (e.g., the second perimeter wall section 98b shown in FIG. 6) may be disposed in at least generally parallel relation. However, such may not be the case after a second transdermal patch 36 has been directed into a corresponding slot 94. In this regard, any appropriate number of flaps 96 could be vertically spaced or staggered within the second compartment 28^{iv} of the containment 90. Generally, these vertically spaced or staggered flaps 96 may provide a tortuous path within the second compartment 28^{iv} which may affect the ability to withdraw second transdermal patches 36 out of the second compartment 28^{iv} through a slot 94.

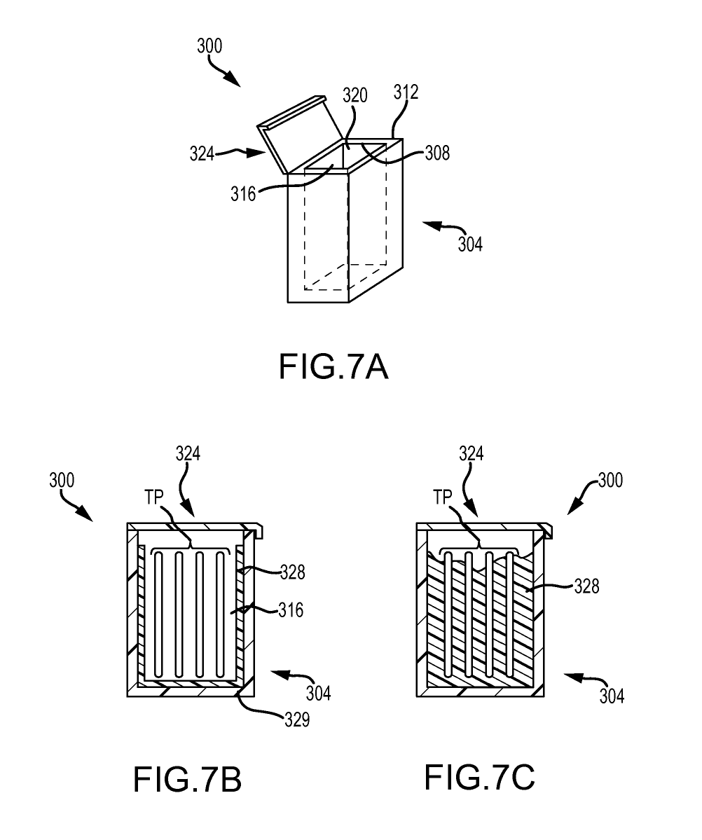

FIG. 7A presents a perspective view of one embodiment of a transdermal patch containment 300 (e.g., case) that includes an encapsulating material of any appropriate type and/or configuration. The containment 300 may include a container body in the form of a shell 304 including inside and outside surfaces 308, 312, in addition to a chamber 316 situated within the shell 304 for holding or containing one or more pharmaceutical products such as one or more new or used transdermal patches TP (e.g., FIGS. 7B and 7C). The inside surface 308 may generally face towards the chamber 316 and the outside surface 312 may generally face away from the containment 300. Access to the chamber 316 may be provided via an access in the form of an opening 320. The containment 300 may also include an access member in the form of a cover 324 (e.g., lid, top) for selectively sealing or closing off the chamber 316 (or otherwise selectively limiting and/or allowing access to the opening 320) and may be interconnected to the shell 304 in any appropriate manner (e.g., pivotally). Although not labeled, the shell 304 and cover 324 may respectively include corresponding locking members to selectively removably attach the cover 324 to the shell 304 and seal or otherwise limit access to the chamber 316.

Turning now to FIG. 7B, a cross-sectional view of the containment 300 is shown, and illustrates a number of transdermal patches TP being received within the chamber 316. As shown, a heat-activated encapsulation component 328 in the form of a sleeve may be appropriately disposed within the chamber 316. Generally, heating the entire containment 300 to at least a melting temperature of the encapsulation component 328, but to a temperature that is less than a melting temperature of the shell 304 and cover 324, will cause the encapsulation component 328 to melt or transition to a different phase (e.g., change from a solid state to a liquid or liquid-like state (e.g., become more flowable)). The encapsulation component 328 is this modified state should come into contact with at least some and more preferably all of the transdermal patches TP contained within the chamber 316. As the containment 300 is allowed to cool, the encapsulation component 328 should again transition to a different phase (e.g., from liquid or liquid-like/flowable state back to a more solidified state). At this time, at least some, and including each of the transdermal patches TP, may be at least partially encapsulated in the encapsulation component 328. Any appropriate material may be used as the encapsulation component 328, including without limitation plastic, wax, adhesive, and all combinations thereof,

The encapsulation component 328 may extend substantially from one side (not labeled) of the chamber 316 to an opposite side (not labeled) of the chamber 316. In other words, the encapsulation component 328 may be sized to have a diameter that is approximately equal to a diameter of the chamber 316 such that the transdermal patches TP may be inserted into or otherwise disposed within the encapsulation component 328. As also shown, the encapsulation component 328 may extend across a bottom surface 329 of the shell 304. Although not illustrated in FIG. 7B, the encapsulation component 328 may also extend from one end (not labeled) of the chamber 316 to an opposite end (not labeled) of the chamber 316 (e.g., the encapsulation component 328 may line at least substantially an entirety of a perimeter of the chamber 316). In this regard, the encapsulation component 328 may be designed to cover or be otherwise be disposed over at least a portion of most or all of the inside surface 308 (e.g., two sides and two ends) of the shell 304. The encapsulation component 328 may be removably disposed in the chamber 316 or may be appropriately attached to the inside surface 308 of the shell 304 (e.g., via adhesives, as part of the manufacturing process of the containment 300).

FIG. 7C is a cross-sectional view of the containment 300 after the cover 324 has been secured to the shell 304 of the containment 300, and after the containment 300 has been heated to at least a melting temperature of the encapsulation component 328 (but less than a melting temperature of the shell 304 and cover 324), and thereafter been allowed to cool for a predetermined period of time. As can be seen, the encapsulation component 328 has melted and thereafter solidified around at least a portion of the transdermal patches TP (e.g., “sandwiched” around) so as to at least partially encapsulate or encase the transdermal patches TP. This should reduce the potential of any further use of the transdermal patches TP. The containment 300 may at this point be appropriately disposed of (e.g., deposited in a trash receptacle) to further reduce the chance of illicit usage of the transdermal patches TP within the containment 300. Although not shown, one or more encapsulation components 328 may be appropriately disposed on an inside surface (not labeled) of the cover 324 which may melt and flow onto the transdermal patches TP and/or locking members or other structures to, after solidifying, further render the transdermal patches TP at least partially unusable and/or non-removably interconnect the cover 324 to the shell 304 to limit access to the chamber 316 and the transdermal patches TP. In some arrangements, the chamber 316 may include one or more ribs or rails attached to the inside surface 308 to define a number of slots, each of which can receive one or more transdermal patches TP or other pharmaceutical products. In this arrangement, one or more encapsulation components 328 may be disposed in each such slot so as to melt around and at least partially encapsulate a transdermal patch TP when the containment 300 has been heated to at least the second melting point.

The embodiments of FIGS. 1-2, 3, 4A-C, 5, and 6 may each utilize an encapsulating material in the manner of the containment 300. Such an encapsulating material may be included in any compartment intended for storing new transdermal patches 34, in any compartment for storing second transdermal patches 36, or both. The flaps discussed above with regard to the embodiments of FIGS. 4A-C, 5, and 6 also may each incorporate an encapsulating material (e.g., a coating; actually forming the flaps from an encapsulating material). Having an encapsulating material within at least part of a compartment for receiving transdermal patches, along with also having one or more flaps that incorporate an encapsulating material, may also be utilized in relation to each of the embodiments of FIGS. 4A-C, 5, and 6. Including a heat-activated adhesive on the flaps discussed above with regard to the embodiments of FIGS. 4A-C, 5, and 6 may also be utilized to facilitate retention of transdermal patches within the associated compartment.

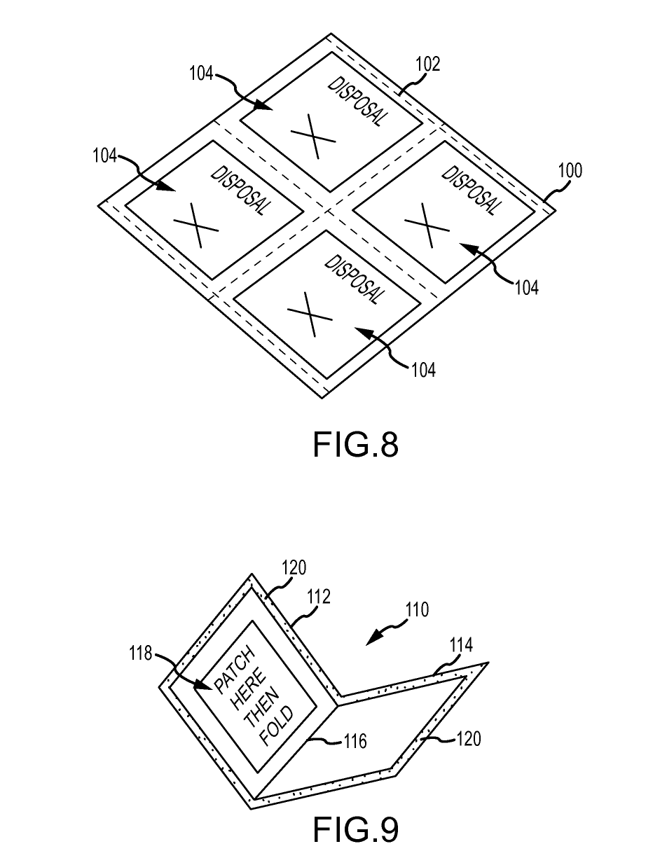

The embodiments of FIGS. 1-6 may be characterized as secondary packaging for transdermal patches (e.g., where “primary packaging” in relation to transdermal patches is typically in the form of a sealed pouch, jacket, or the like). Additional features may be utilized to further facilitate the disposal of used transdermal patches. One embodiment of what may be characterized as a transdermal patch (“TP”) disposal member is illustrated in FIG. 8 and is identified by reference numeral 100. The TP disposal member 100 generally includes a plurality of predefined regions or zones 104. Each zone 104 accommodates an individual transdermal patch, and each zone 104 may include any appropriate labeling/messaging as desired/required. Preferably a bond exists between the TP disposal member 100 and each transdermal patch positioned thereon (e.g., within a given zone 104). A new TP disposal member 100 could be stored in secondary packaging that also includes new transdermal patches (e.g., first transdermal patches 34), or a new TP disposal member 100 could be provided separately from any such secondary packaging.

The TP disposal member 100 may be in the form of a sheet or substrate formed from any appropriate material or combination of materials, and may be of any appropriate structural configuration. The TP disposal member 100 may also be of any appropriate size, and may include any appropriate number of zones 104. Each zone 104 may include an appropriate adhesive. This adhesive could occupy the entirety of each particular zone 104, or could be disposed in any appropriate arrangement within each particular zone 104 (e.g., disposed about an annular perimeter of each zone 104, such as in the case of the embodiment of FIG. 9 that is discussed below). When an adhesive is used by the TP disposal member 100, it may be desirable to include a release liner or film 102 of any appropriate type. Removal of the liner 102 over a particular zone 104 allows a transdermal patch to be positioned on the TP disposal member 100 within this zone 104. Having the TP disposal member 100 incorporate an adhesive in each zone 104 may further reduce the potential that a transdermal patch could be removed from the TP disposal member 100. However, the remaining adhesive on a transdermal patch could be used to establish a bond with the TP disposal member 100.

The TP disposal member 100 could be disposed within secondary packaging in the form illustrated in FIG. 8. However, the TP disposal member 100 could be configured such that an individual subsection, that includes a single zone 104, could be removable from a remainder of the TP disposal member 100. These individual subsections could then be disposed within secondary packaging in accordance with the foregoing for disposal purposes. For instance, such individual subsections could be directed into the opening 30 and into the second compartment 28 of the transdermal patch container 10 of FIGS. 1-2, through a slot 44 and into the second compartment 28ⁱ of the transdermal patch container 10ⁱ of FIG. 3, etc.

One embodiment of what may be characterized as an individual transdermal patch disposal card is illustrated in FIG. 9 and is identified by reference numeral 110. One or more disposal cards 110 could be stored in secondary packaging that also includes new transdermal patches, or one or more disposal cards 110 could be provided separately from any such secondary packaging.

The transdermal patch disposal card 110 may include a first section 112, a second section 114, and a fold line 116 therebetween. The fold line 116 could be pre-existing or predefined, or the fold line 116 could be created by a user folding the disposal card 110 in half after placing a used transdermal patch thereon. In the illustrated embodiment, the first section 112 may include labeling/messaging 118 as desired/required. Any appropriate labeling or messaging may be provided on the disposal card 110.

An appropriate adhesive 120 may be included on at least part of at least one of the first section 112 and the second section 114. A release liner or film (not shown) may then be used by the disposal card 110 so as to cover the adhesive 120 until needed for receiving a transdermal patch. The adhesive 120 could occupy the entirety of one or both of the sections 112, 114 (not shown), or could be disposed in any appropriate arrangement on one or both of the sections 112, 114. In the illustrated embodiment, adhesive 120 is disposed only about the perimeter of each of the sections 112, 114. Although remaining adhesive on a transdermal patch could provide a suitable bond between the transdermal patch and the disposal card 110 (e.g., when positioning the adhesive-side of the transdermal patch directly against the disposal card 110), providing adhesive 120 on at least one of the first section 112 and the second section 114 may be utilized to encapsulate a transdermal patch within the disposal card 110 (e.g., when folding the first section 112 relative to the second section 114). This may further enhance the disposal of transdermal patches. Although the transdermal patch disposal card 110 is illustrated as accommodating only a single transdermal patch, it may be sized to accommodate any appropriate number of transdermal patches (e.g., before being folded to capture one or more used transdermal patches therebetween).

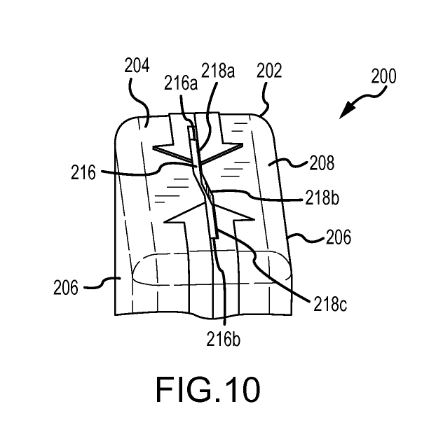

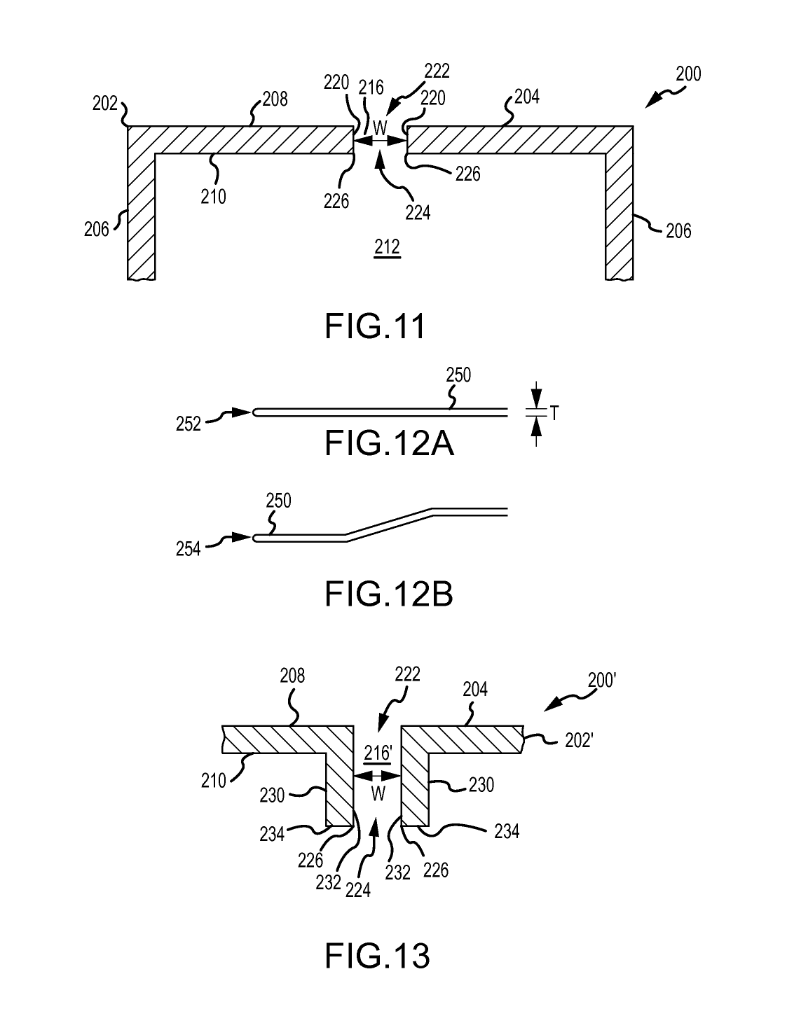

One embodiment of a transdermal patch disposal device is illustrated in FIGS. 10 and 11, and is identified by reference numeral 200. The transdermal patch disposal device 200 includes a containment 202 that incorporates a patch disposal slot 216 for accessing an internal chamber 212. The containment 200 may be of any appropriate size, shape, and/or configuration, and furthermore may be formed from any appropriate material or combination of materials. In one embodiment, the containment 202 is formed from an appropriate disposable plastic or plastic-like material. In the illustrated embodiment, the containment 202 includes a first end wall 204 and a pair of sidewalls 206. For instance, the containment 202 may be in the form of a hollow box or the like. Other configurations may be appropriate.

The containment 202 may incorporate the patch disposal slot 216 at any appropriate location. In the illustrated embodiment, the patch disposal slot 216 is included on the first end wall 204. The first end wall 204 may define an upper end of the containment 202 when disposed on an appropriate supporting surface. Regardless of where the patch disposal slot 216 is incorporated by the containment 202, the patch disposal slot 216 extends from an exterior surface 208 of the containment 202 to an interior surface 210 of the containment 202.

The patch disposal slot 216 is of a non-linear configuration in the case of the patch disposal device 200.

Consider the case where the patch disposal slot 216 includes a first slot end 216a and an oppositely disposed second slot end 216b. The length dimension of the patch disposal slot 216 may be characterized as proceeding from the first slot end 216a to the second slot end 216b. At least part of the patch disposal slot 216, proceeding from the first slot end 216a to the second slot and 216b, is other than collinear with another part of the patch disposal slot 216. That is, the patch disposal slot 216 does not proceed along a linear or axial path all the way from its first slot end 216a to its second slot end 216b.

Any appropriate non-linear configuration for a length dimension of the patch disposal slot 216 may be utilized by the transdermal patch disposal device 200. Representative non-linear configurations include without limitation having a length dimension of the patch disposal slot 216 being at least generally S-shaped (the configuration shown in FIG. 10) or C-shaped (not shown, but where at least part of the patch disposal slot 216 is curved proceeding along at least part of its length dimension). In the illustrated embodiment, the noted S-shaped configuration of the patch disposal slot 216 is realized by the slot 216 being defined by a first slot section 218a, a second slot section 218b, and a third slot section 218c. Each of the slot sections 218a-c may also be characterized as a slot length segment.

The entirety of the patch disposal slot 216 is defined by the slot sections 218a-c in the illustrated embodiment, where the second slot section 218b is located between the first slot section 218a and the third slot section 218c. The second slot section 218b is disposed other than collinear with at least one of the first slot section 218a and the third slot section 218c. In the illustrated embodiment, the second slot section 218b is disposed other than collinear with each of the first slot section 218a and the third slot section 218c. Each of the slot sections 218a-c may be of any appropriate configuration proceeding along their respective length dimensions. In the illustrated embodiment, each of the individual slot sections 218a-c is linearly or axially extending along their respective length dimension. The first slot section 218a and the third slot section 218c may be disposed in parallel relation, with the second slot section 218b interconnecting adjacent most ends of the first slot section 218a and third slot section 218c.

Referring now to FIG. 11, the patch disposal slot 216 is defined by a pair of slot sidewalls 220. The vertical extent of the slot sidewalls 220 in the view shown in FIG. 11 is defined by the wall thickness of the containment 202 through which the patch disposal slot 216 extends. The intersection of each of the slot sidewalls 220 with the exterior surface 208 may be “rounded off” to facilitate directing transdermal patches into/through the transdermal patch disposal slot 216.

The intersection of each slot sidewall 220 with the interior surface 210 of the containment 202 may define an edge 226 (e.g., in the form of a knife edge). In one embodiment, each edge 226 is defined by an intersection of planar surfaces. Incorporating edges 226 (e.g., knife edges) at the interior end 224 of the patch disposal slot 216 should reduce the potential of being able to withdraw a transdermal patch out of the internal chamber 212 back through the patch disposal slot 216. Incorporating one or more edges 226 at this end of patch disposal slot 216 (e.g., as opposed to utilizing rounded corners) may provide a “mechanical catch” that may impede removing a transdermal patch out from the internal chamber 212 through the patch disposal slot 216 (e.g., a transdermal patch may become “snagged” on one or more of such edges 226 if someone attempts to withdraw a transdermal patch out of the internal chamber 212 through the patch disposal slot 216). This feature then should enhance the disposability of transdermal patches by using the transdermal patch disposal device 200. A “sharp edge” feature along the slot 216 may be used by other transdermal patch disposal devices 200′, 200″, 200′″ that are addressed herein.

A spacing between the slot sidewalls 220 defines a width W for the patch disposal slot 216 as also shown in FIG. 11. The width W of the patch disposal slot 216 may be constant proceeding along its length dimension (e.g., proceeding from its first slot end 216a to its second slot end 216b), although such may not be required in all instances. In one embodiment, the width W of the patch disposal slot 216 is only slightly larger than a thickness of a transdermal patch as it is being directed through the patch disposal slot 216 and into the internal chamber 212. A transdermal patch passes an exterior end 222 of the patch disposal slot 216 and then an interior end 224 of the patch disposal slot 216 as the transdermal patch passes through the slot 216 and into the internal chamber 212. A “narrow slot” feature may be used by other transdermal patch disposal devices 200′, 200″, 200′″ that are addressed herein.

FIGS. 12A-B illustrate a transdermal patch 250 that may be disposed of by being directed through the patch disposal slot 216 and into the internal chamber 212 of the transdermal patch disposal device 200 of FIGS. 10-11. In the illustrated embodiment, the transdermal patch 250 is disposed in a folded configuration (e.g., where the transdermal patch 250 is folded over onto itself). The transdermal patch 250 may be retained in this folded-over configuration, for instance by adhesive that may remain on the transdermal patch 250 and that is used to attach the transdermal patch 250 to a patient. Generally, the transdermal patch 250 has a thickness T in the illustrated folded-over configuration. In one embodiment, the width W of the patch disposal slot 216 is no more than about 18% larger than the thickness T of the transdermal patch 250 in a form for being directed through the patch disposal slot 216 and into the internal chamber 212. Having the width W of the patch disposal slot 216 being only slightly larger than the thickness T of the transdermal patch 250 (in a form for being directed through the patch disposal slot 216 and into the internal chamber 212), should reduce the potential of being able to withdraw the transdermal patch 250 out of the internal chamber 212 through the patch disposal slot 216. This then should enhance the disposability of transdermal patches by using the transdermal patch disposal device 200.

The transdermal patch 250 is also of a first configuration 252 in FIG. 12A (e.g., an unbiased condition or state). A second configuration 254 for the transdermal patch 250 is shown in FIG. 12B. The transdermal patch 250 may be bent, twisted, deformed, or manipulated from the first configuration 252, into the second configuration 254, for purposes of (or by) directing the transdermal patch 250 through the patch disposal slot 216. Generally, the configuration of the transdermal patch 250 may be altered to at least generally correspond to the configuration of the patch disposal slot 216 proceeding along its length dimension, all for purposes of positioning the patch 250 within the internal chamber 212. In any case, the transdermal patch 250 may pass from the exterior end 222 of the patch disposal slot 216 and then to its interior end 224 when being directed through the slot 216 for storage within the internal chamber 212.

Changing the transdermal patch 250 from the first configuration 252 to the second configuration 254 may be characterized as elastically deforming the transdermal patch 250, storing energy within the transdermal patch 250, or the like. The elasticity of the transdermal patch 250, resiliency of the transdermal patch 250, and/or stored energy within the transdermal patch 250 provides a force that will attempt to change the transdermal patch 250 back to its first configuration 252 when the configuration-altering force is removed from the transdermal patch 250. In this regard, prior to or as the transdermal patch 250 is being directed through the patch disposal slot 216, the transdermal patch 250 may be of a configuration that at least generally matches the configuration of the patch disposal slot 216 in relation to its length dimension (e.g., the second configuration 254 of FIG. 12B). Changing the transdermal patch 250 into such a configuration will store at least some energy within the transdermal patch 250 (e.g., generate internal stresses). Once the transdermal patch 250 has progressed entirely through the patch disposal slot 216 and entered the internal chamber 212, the configuration-changing force will be removed from the transdermal patch 250 and the elasticity, resiliency, and/or stored energy will attempt to change the transdermal patch 250 back to its original first configuration 252. No external forces need be exerted on the transdermal patch 250 to have the transdermal patch 250 at least attempt to “spring back” from its second configuration 254. Although the transdermal patch 250 could change all the way back to its first configuration 252 (e.g., via spring-back) once within the internal chamber 212, the elasticity, resiliency, and/or stored energy may only change the transdermal patch 250 back to a configuration that is somewhere between the second configuration 254 and the first configuration 252. In either case, having the transdermal patch 250 “spring back” to at least a certain extent upon entering the internal chamber 212 should reduce the potential of being able to withdraw the transdermal patch 250 out of the internal chamber 212 back through the patch disposal slot 216. This then should enhance the disposability of transdermal patches by using the transdermal patch disposal device 200.

A variation of the transdermal patch disposal device 200 of FIGS. 11-12 is presented in FIG. 13 and is identified by reference numeral 200′. Corresponding components between the embodiment of FIGS. 11 and 13 are identified by common reference numerals. Those corresponding components that differ in at least some respect are identified by a “single prime” designation. The primary difference between the transdermal patch disposal device 200′ of FIG. 13 and the transdermal patch disposal device 200 of FIGS. 11-12 is the inclusion of a pair of projections 230 by the containment 202′ of the transdermal patch disposal device 200′. Each of the projections 230 extend from an interior surface 210 of the containment 202′, and may be characterized as cantilevers (i.e., having a supported end (at the interior surface 210) and an oppositely disposed and free/unsupported end).

The projections 230 are spaced from each other and define at least a portion of the patch disposal slot 216′. Generally, the projections 230 may be characterized as increasing the depth of the patch disposal slot 216′, or the spacing between the exterior end 222 and the exterior end 224 of the patch disposal slot 216′, compared to the embodiment of FIGS. 10-11. Each projection 230 may also incorporate an edge 226 (e.g., a knife edge) at the interior end 224 of the patch disposal slot 216′ (e.g., defined by an intersection between a projection sidewall 232 and a corresponding projection end wall 234, each of which may be planar surfaces). Increasing the depth of the patch disposal slot 216′, alone or in combination with incorporating a pair of spaced edges 226 (e.g., knife edges) at the interior end 224 of the patch disposal slot 216′, should reduce the potential of being able to withdraw a transdermal patch out of the internal chamber 212 back through the patch disposal slot 216′. This then should enhance the disposability of transdermal patches by using the transdermal patch disposal device 200. The projections 230 may also be utilized by the other transdermal patch disposal devices 200, 200″, and 200′″ addressed herein.

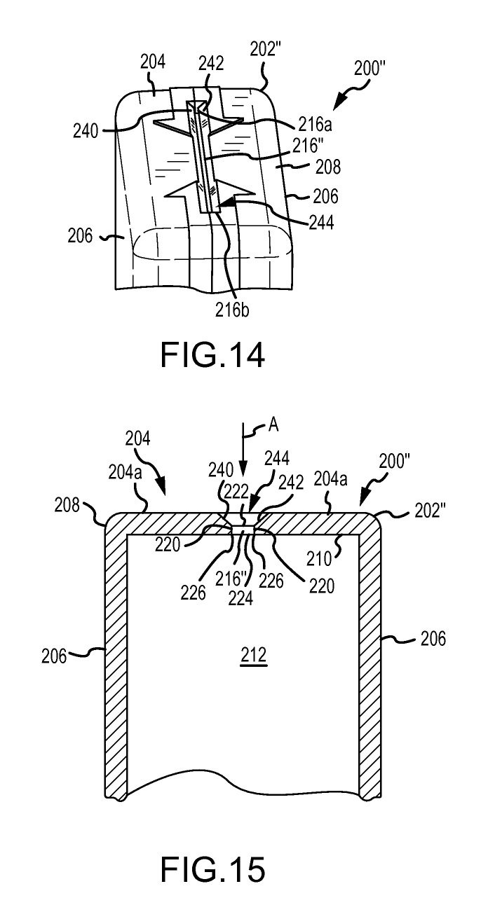

Another variation of the transdermal patch disposal device 200 of FIGS. 11-12 is presented in FIGS. 14-15 and is identified by reference numeral 200″. Corresponding components between the embodiments of FIGS. 11 and 14 are identified by common reference numerals. Those corresponding components that differ in at least some respect are identified by a “double prime” designation. There are two distinctions between the embodiments of FIGS. 11 and 14. One is that the containment 202″ of the transdermal patch disposal device 200″ shown in FIGS. 14-15 includes an axially or linearly extending (along its length dimension) patch disposal slot 216″. Another is that the exterior surface 208 of the containment 202″ includes a recess 244. The patch disposal slot 216″ is located within this recess 244.

The recess 244 may be characterized as being at least generally v-shaped in a cross-sectional view taken perpendicular to the length dimension (as well as coplanar to both the width and depth dimensions) of the patch disposal slot 216″ (e.g., the view shown in FIG. 15). The width of the recess 244 (such a width being in the left-to-right direction in the view shown in FIG. 15) is progressively reduced proceeding in the direction of the patch disposal slot 216″ (i.e., in the direction of the arrow A in FIG. 15).

The above-noted recess 244 may be collectively defined by a pair of chamfers 240, 242. The patch disposal slot 216″ may be characterized as being located between a first chamfer 240 and a second chamfer 242 (including where the entirety of the patch disposal slot 216″ is located between the chamfers 240, 242). The first chamfer 240 may lead to the patch disposal slot 216″ and may be positioned on a first side of the patch disposal slot 216″, while the second chamfer 242 may lead to the patch disposal slot 216″ and may be positioned on a second side of the patch disposal slot 216″ (e.g., where the first and second sides of the patch disposal slot 216″ are oppositely disposed). The chamfers 240, 242 may each proceed along an entirety of a length of the patch disposal slot 216″.

The first chamfer 240 and second chamfer 242 may each be characterized as angled surfaces. The chamfers 240, 242 may be disposed in different orientations. The first chamfer 240 may be the mirror image of the second chamfer 242. In one embodiment, the chamfers 240, 242 are each planar surfaces. Other contours may be appropriate.

The chamfers 240 and 242 may extend from a common portion of the exterior surface 208, for instance a first wall section 204a of the first end wall 204 that also includes the recess 244. This first wall section 204a may be in the form of a planar surface. In any case, each chamfer 240, 242 is disposed other than perpendicularly to the first wall section 204a (e.g., the included angle between each chamfer 240, 242 and the first wall section 204a may be more than 180° and less than 270°). Each chamfer 240, 242 is also disposed other than coplanar with the corresponding slot sidewall 220. The chamfers 240, 242 may define an offset between the first wall section 204a and the exterior end 222 of the patch disposal slot 216″. In the illustrated embodiment and in the view shown in FIG. 15, each chamber 240, 242 extends both in the vertical dimension (i.e., downwardly) and in the horizontal dimension (e.g., to the left or the right) proceeding from the first wall section 204a to the exterior end 222 of the patch disposal slot 216″.

As previously noted, the transdermal patch disposal device 200″ of FIGS. 14-15 may utilize the projections 230 discussed above in relation to the embodiment of FIG. 13. These projections 230 would be spaced to define at least part of the “depth” of the patch disposal slot 216″, and could be used to increase the depth of the patch disposal slot 216″ compared to the FIG. 15 configuration.

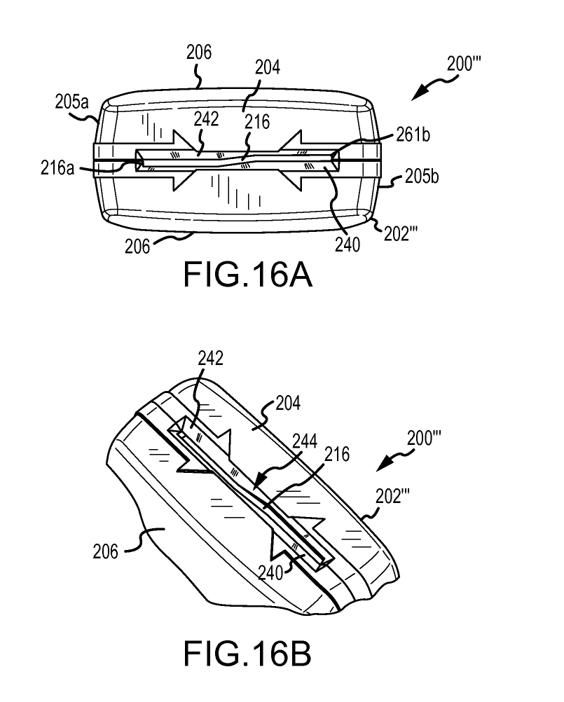

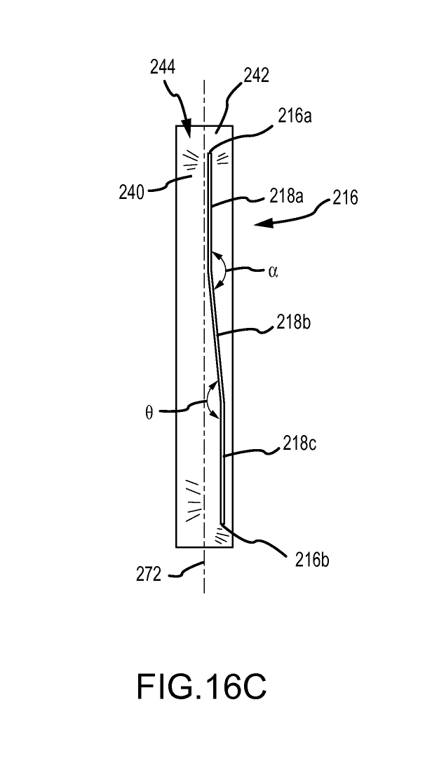

Another variation of the transdermal patch disposal device 200 of FIGS. 11-12 is presented in FIGS. 16A-C and is identified by reference numeral 200′″. Corresponding components between the embodiments of FIGS. 11 and 16A-B are identified by common reference numerals. Those corresponding components that differ in at least some respect are identified by a “triple prime” designation. Generally, the transdermal patch disposal device 200′″ may be viewed as a combination of the embodiments of FIGS. 11 and 14. That is, the containment 202′″ in FIGS. 16A-C includes the patch disposal slot 216 from the embodiment of FIGS. 11-12, along with the recess 244 from the embodiment of FIGS. 14-15 (e.g., the first chamfer 240 and second chamfer 242). The patch disposal slot 216 is also located within the recess 244 for the transdermal patch disposal device 200′″. A variation of the embodiment of FIGS. 16A and 16B would be to incorporate separate chamfers for each of the slot sections 218a-c along each side thereof. The transdermal patch disposal device 200′″ of FIGS. 16A-B may utilize the projections 230 discussed above in relation to the embodiment of FIG. 13. These projections 230 would be spaced to define at least part of the “depth” of the patch disposal slot 216 and could be used to increase the depth of the patch disposal slot 216 compared to the configuration shown in FIGS. 16A-C.

The length dimension of the patch disposal slot 216 coincides with the spacing between end walls 205a, 205b of the containment 202′″ of the transdermal patch disposal device 200′″ (as it also does in the case of the embodiments of FIGS. 10 and 14 discussed above). In relation to the illustrated configuration of the containment 202′″: 1) the end wall 205a may be characterized as its front wall or end; 2) the end wall 205b may be characterized as its rear wall or end; and 3) the end wall 204 may be characterized as its upper wall or end. In any case, the first slot section 218a and the third slot section 218c may be disposed in at least substantially parallel relation (more specifically, their respective length dimensions), with the second slot section 218b connecting adjacent ends of the slot sections 218a, 218c and with the second slot section 218b being disposed in non-parallel relation to each of the first slot section 218a and the third slot section 218c (more specifically, in relation to their respective length dimensions). The first slot section 218a and the third slot section 218c are also disposed in at least substantially parallel relation to a reference axis 272 that extends between corresponding portions of the end walls 205a, 205b of the containment 202′″, while the second slot section 218b is disposed in non-parallel relation to this same reference axis 272. As such, the magnitude of an included angle a and the magnitude of an included angle θ may be at least substantially equal (where the included angle a is that included angle between the first slot section 218a and the second slot section 218b which is less than 180°, and where the included angle θ is that included angle between the second slot section 218b and the third slot section 218c which is less than 180°).

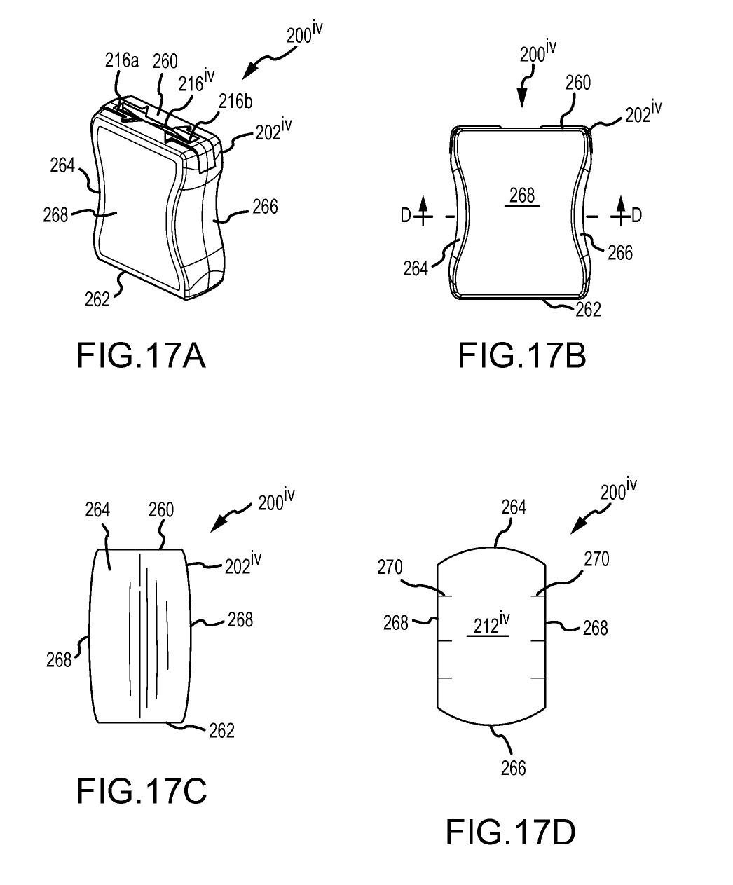

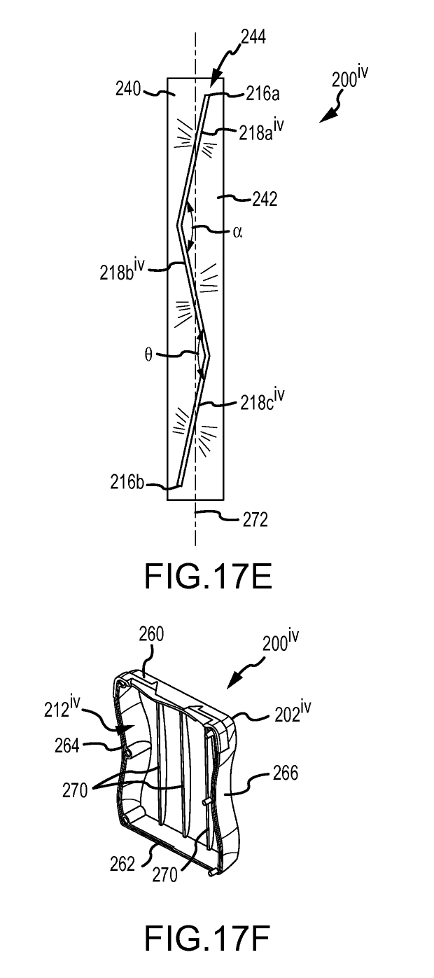

Another embodiment of a transdermal patch disposal device is presented in FIGS. 17A-F and is identified by reference numeral 200^{iv}. Generally, the transdermal patch disposal device 200^{iv} uses one or more of a different exterior shaping for its containment, a different orientation for the transdermal patch disposal slot from that shown in the embodiment of FIGS. 16A-C, and an additional internal patch retention feature. Corresponding components between the above-noted embodiments that differ in at least some respect may be identified by a superscripted “iv” designation in the embodiment of FIGS. 17A-F.

The transdermal patch disposal device 200^{iv} may utilize a different external shaping for its containment 202^{iv} compared to the above-noted embodiments. The containment 202^{iv} includes an end wall 260 (e.g., an upper wall or end) and an oppositely disposed end wall 262 (e.g., a lower wall or end), an end wall 264 (e.g., a front wall or end) and an oppositely disposed end wall 266 (e.g., a rear wall or end), and a pair of oppositely disposed sidewalls 268. Hereafter, the containment 202^{iv} will be described with regard to it being maintained in a position where the end wall 260 projects upwardly, or so as to define an upper wall for the containment 202^{iv}. However, the containment 202^{iv} may of course be maintained in any orientation when positioning a transdermal patch therein (although the end wall 260 may still be referred to as “upper wall 260”).

Each of the sidewalls 268 of the containment 202^{iv} may be characterized as being at least generally convex or as being bulged outwardly in relation to the internal chamber 212^{iv} of the containment 202^{iv} (e.g., FIG. 17C). In one embodiment, this convexity or curvature is in a dimension coinciding with the spacing between the upper wall 260 and the lower wall 262 of the containment 202^{iv}. All or a portion of each sidewall 268 could include the noted convex configuration on an exterior of the containment 202^{iv}.

The front wall 264 and the rear wall 266 each may be characterized as being at least generally concave or as including an external recess or notch in one dimension and in relation to the internal chamber 212^{iv} of the containment 202^{iv} (e.g., FIGS. 17A and 17B). In one embodiment, this concavity or curvature is in a dimension coinciding with the spacing between the upper wall 260 and the lower wall 262 of the containment 202^{iv}. All or a portion of each of the front wall 264 and the rear wall 266 could include the noted concave configuration on an exterior of the containment 202^{iv}.

The front wall 264 and the rear wall 266 each may be characterized as being at least generally convex or as being bulged outwardly in another dimension and in relation to the internal chamber 212^{iv} of the containment 202^{iv} (e.g., FIG. 17D). In one embodiment, this convexity or curvature is in a dimension coinciding with the spacing between the two oppositely disposed sidewalls 268. All or a portion of each of the front wall 264 and the rear wall 266 could include the noted convex configuration on an exterior of the containment 202^{iv}. In the case where the front wall 264 and the rear wall 266 include a concavity in a dimension coinciding with the spacing between the upper wall 260 and the lower wall 262 (a first dimension), along with a convexity in a dimension coinciding with the spacing between the two oppositely disposed sidewalls 268 (a different, second dimension, and which may be orthogonal to the noted first dimension), the front wall 264 and the rear wall 266 may each be characterized as being at least generally saddle-shaped or of a saddle-like configuration (e.g., including curvatures in two different dimensions).

The above-noted configuration of the containment 202^{iv} may facilitate handling of the same by a user when directing a transdermal patch through the transdermal patch disposal slot 216^{iv} and into its internal chamber 212^{iv}. The above-noted features on the exterior of the containment 202^{iv} may allow a user to comfortably and/or efficiently grip the containment 202^{iv} when directing a transdermal patch through the transdermal patch disposal slot 216^{iv} and into its internal chamber 212^{iv}. Any of the noted external “shaping” features of the containment 202^{iv} may be used by any one or more of the above-described embodiments.

The patch disposal slot 216^{iv} for the transdermal patch disposal device 200^{iv} is in a different orientation compared to the above-discussed embodiments. The length dimension of the patch disposal slot 216^{iv} coincides with the spacing between the front wall 264 and the rear wall 266 of the containment 202^{iv} for the transdermal patch disposal device 200^{iv}. A reference axis 272 extends between corresponding portions of the front wall 264 and rear wall 266 of the containment 202^{iv}. Referring primarily to FIG. 17E, neither the first slot section 218a^{iv}, the second slot section 218b^{iv}, nor the third slot section 218c^{iv} has its length dimension disposed in at least substantially parallel relation to this reference axis 272. That is, the length dimension for each of the first slot section 218a^{iv}, the second slot section 218b^{iv}, and the third slot section 218c^{iv} is disposed in non-parallel relation to this reference axis 272. This orientation for the patch disposal slot 216^{iv} could be used in relation to any of the other embodiments described herein. Although the patch disposal slot 216^{iv} is illustrated as being disposed within a recess 244, other embodiments may not utilize such a recess 244 on the upper wall 260.

The first slot section 218a^{iv} and the third slot section 218c^{iv} may be disposed in at least substantially parallel relation (more specifically, their respective length dimensions), with the second slot section 218b^{iv} connecting adjacent ends of the slot sections 218a^{iv}, 218c^{iv} and with the second slot section 218b^{iv} being disposed in non-parallel relation to each of the first slot section 218a^{iv} and the third slot section 218c^{iv} (more specifically, in relation to their respective length dimensions). As such, the magnitude of an included angle a and the magnitude of an included angle θ may be at least substantially equal (where the included angle a is that included angle between the first slot section 218a^{iv} and the second slot section 218b^{iv} which is less than 180°, and where the included angle θ is that included angle between the second slot section 218b^{iv} and the third slot section 218c^{iv} which is less than 180°).

The transdermal patch disposal device 200^{iv} may utilize one or more ribs 270 that are disposed within the internal chamber 212^{iv} of its containment 202^{iv}. Utilizing one or more ribs 270 may provide structural reinforcement for the containment 202^{iv}. Utilizing one or more ribs 270 may reduce the potential of being able to withdraw a transdermal patch out of the internal chamber 212^{iv} through the patch disposal slot 216^{iv}. One or more of the other features addressed herein that are directed to attempting to reduce the potential of being able to withdraw a transdermal patch 250 out of the internal chamber 212^{iv} through the patch disposal slot 216^{iv} may be utilized as well (e.g., having the “entry” into the slot 216^{iv} (from the side of the internal chamber 212^{iv}) include one or more edges; incorporating the projections 230).

Each rib 270 may extend from an interior surface of a sidewall 268 and into the internal chamber 212^{iv} of the containment 202^{iv}. The length dimension of each rib 270 may at least generally coincide with the spacing between the upper wall 260 (that incorporates the patch disposal slot 216iv) and the oppositely disposed lower wall 262 of the containment 202^{iv}. Any appropriate number of ribs 270 may be utilized in relation to the internal chamber 212^{iv}. In the illustrated embodiment, each rib 270 associated with one of the sidewalls 268 includes a corresponding rib 270 on the opposite sidewall 268 (e.g., the internal surfaces of the sidewalls 268 may be the mirror image of each other at least in relation to these ribs 270). Such may not be required in all instances.

Various embodiments are described herein that utilize a slot through which a transdermal patch is directed for purposes of and/or in preparation for disposal of the corresponding containment. It may be desirable to place tape over each such slot prior to disposing of the containment (e.g., prior to placing the containment in the trash). One or more pieces of tape could be utilized. Appropriate information could be printed on any such tape, for instance information for purposes of complying with regulatory requirements (e.g., identifying the name of the active ingredient(s) associated with the transdermal patches).

The foregoing description of the present invention has been presented for purposes of illustration and description. Furthermore, the description is not intended to limit the invention to the form disclosed herein. Consequently, variations and modifications commensurate with the above teachings, and skill and knowledge of the relevant art, are within the scope of the present invention. The embodiments described hereinabove are further intended to explain best modes known of practicing the invention and to enable others skilled in the art to utilize the invention in such, or other embodiments and with various modifications required by the particular application(s) or use(s) of the present invention. It is intended that the appended claims be construed to include alternative embodiments to the extent permitted by the prior art.

What is claimed:

1. A transdermal patch disposal device comprising:
a containment;
an internal chamber within said containment;
a patch disposal slot that is other than linear, that extends completely through said containment, and that accesses said internal chamber; and
at least one transdermal patch disposed within said internal chamber, wherein said at least one transdermal patch comprises a first transdermal patch.

2. The disposal device claim 1, wherein said containment comprises a first wall, and wherein said first wall comprises said patch disposal slot.

3. The disposal device of claim 2, wherein said first wall comprises an upper end of said containment.

4. The disposal device of claim 1, wherein said patch disposal slot is other than linear proceeding along an entirety of a length dimension of said patch disposal slot.

5. The disposal device of claim 1, wherein said patch disposal slot comprises first and second slot ends, wherein at least one part of said patch disposal slot positioned along a path proceeding from said first slot end to said second slot end is other than linearly aligned relative to at least one other part of said patch disposal slot positioned along said path proceeding from said first slot end to said second slot end.

6. The disposal device of claim 1, wherein said patch disposal slot comprises first, second, and third segments, with said second segment being located between said first and third segments, wherein said second segment connects said first and third segments, and wherein said second segment is other than axially aligned with each of said first and third segments.

7. The disposal device of claim 6, wherein said first and third segments are disposed in at least substantially parallel relation.

8. The disposal device of claim 6, wherein said containment comprises first and second end walls that are oppositely disposed, and third and fourth end walls that are oppositely disposed, wherein said first end wall comprises said patch disposal slot, wherein said first segment is located between said third end wall and said second segment, wherein said third segment is located between said fourth end wall and said second segment, wherein a reference axis extends between corresponding portions of said third and fourth end walls, wherein a length dimension of said first and third segments is at least substantially parallel with said reference axis, and wherein a length dimension of said second segment is non-parallel with said reference axis.

9. The disposal device of claim 6, wherein said containment comprises first and second end walls that are oppositely disposed, and third and fourth end walls that are oppositely disposed, wherein said first end wall comprises said patch disposal slot, wherein said first segment is located between said third end wall and said second segment, wherein said third segment is located between said fourth end wall and said second segment, wherein a reference axis extends between corresponding portions of said third and fourth end walls, and wherein a length dimension of each of said first, second, and third segments is non-parallel with said reference axis.

10. The disposal device of claim 6, wherein said second segment extends along an axial path between adjacent most ends of said first and third segments.

11. The disposal device of claim 6, wherein each of said first, second, and third segments extend axially in their respective length dimension.

12. The disposal device of claim 11, wherein a first included angle exists between said first and second segments and is less than 180°, wherein a second included angle exists between said second and third segments and is less than 180°, and wherein said first and second included angles are of a common magnitude.

13. The disposal device of claim 1, wherein a width of said patch disposal slot is of a first magnitude, wherein a thickness of said first transdermal patch is of a second magnitude, and wherein said first magnitude is only slightly larger than said second magnitude.

14. The disposal device of claim 13, wherein said first magnitude is no more than about 18% larger than said second magnitude.

15. The disposal device of claim 1, wherein said patch disposal slot extends from an exterior surface of said containment to an interior surface of said containment, and wherein an intersection between said interior surface and each slot sidewall defines an edge.

16. The disposal device of claim 15, wherein each said edge is a knife edge.

17. The disposal device of claim 1, further comprising:
a first chamfer leading to said patch disposal slot and positioned on a first side of said patch disposal slot; and
a second chamfer leading to said patch disposal slot and positioned on a second side of said patch disposal slot.

18. The disposal device of claim 17, wherein said first and second chamfers each proceed along an entirety of a length of said patch disposal slot.

19. The disposal device of claim 17, wherein said first and second chamfers are disposed in different orientations.

20. The disposal device of claim 17, wherein said first chamfer is a mirror image of said second chamfer.

21. The disposal device of claim 17, wherein each of said first and second chamfers are planar surfaces.

22. The disposal device of claim 17, wherein each of said first and second chamfers extend from a common first wall section of said containment to said patch disposal slot, and wherein each of said first and second chamfers extend between said first wall section and an exterior end of said patch disposal slot at an angle relative to said first wall section.

23. The disposal device of claim 22, wherein said containment further comprises first and second slot sidewalls disposed in spaced relation, wherein said first chamfer extends from said first wall section to said first slot sidewall, wherein said first chamfer is in a different orientation than each of said first wall section and said first slot sidewall, wherein said second chamfer extends from said first wall section to said second slot sidewall, and wherein said second chamfer is in a different orientation than each of said first wall section and said second slot sidewall.

24. The disposal device of claim 1, wherein said containment comprises a recess, and wherein said patch disposal slot is disposed within said recess.

25. The disposal device of claim 24, wherein a width of said recess is progressively reduced proceeding in a direction of said patch disposal slot.

26. The disposal device of claim 1, further comprising:
first and second projections that extend within said internal chamber, wherein said patch disposal slot extends between said first and second projections, and wherein said first and second projections collectively define at least part of a depth of said patch disposal slot.

27. The disposal device of claim 26, wherein said containment comprises a first interior surface, and wherein said first and second projections extend from said first interior surface.

28. The disposal device of claim 1, wherein said first transdermal patch is maintained in a folded configuration within said internal chamber.

29. The disposal device of claim 1, wherein said containment comprises first and second end walls that are oppositely disposed, third and fourth end walls that are oppositely disposed, and first and second sidewalls that are oppositely disposed, wherein said first end wall comprises said patch disposal slot, wherein a length dimension of said patch disposal slot coincides with a spacing between said third and fourth end walls, wherein each of said first and second sidewalls intersects with each of said first, second, third, and fourth end walls, and wherein each of said third and fourth end walls comprises a surface that is concave in a dimension coinciding with a spacing between said first and second end walls.

30. The disposal device of claim 1, wherein said containment comprises first and second end walls that are oppositely disposed, third and fourth end walls that are oppositely disposed, and first and second sidewalls that are oppositely disposed, wherein said first end wall comprises said patch disposal slot, wherein a length dimension of said patch disposal slot coincides with a spacing between said third and fourth end walls, wherein each of said first and second sidewalls intersects with each of said first, second, third, and fourth end walls, and wherein each of said first and second sidewalls comprise a surface that is convex in a dimension coinciding with a spacing between said first and second end walls.

31. The disposal device of claim 1, wherein said containment comprises first and second end walls that are oppositely disposed, third and fourth end walls that are oppositely disposed, and first and second sidewalls that are oppositely disposed, wherein said first end wall comprises said patch disposal slot, wherein a length dimension of said patch disposal slot coincides with a spacing between said third and fourth end walls, wherein each of said first and second sidewalls intersects with each of said first, second, third, and fourth end walls, wherein each of said third and fourth end walls comprises a surface that is concave in a dimension coinciding with a spacing between said first and second end walls, and wherein each of said first and second sidewalls comprises a surface that is convex in a dimension coinciding with a spacing between said first and second end walls.

32. The disposal device of claim 1, wherein said internal chamber comprises at least one rib.

33. The disposal device of claim 1, wherein said containment comprises first and second end walls that are oppositely disposed, third and fourth end walls that are oppositely disposed, and first and second sidewalls that are oppositely disposed, wherein said first end wall comprises said patch disposal slot, wherein a length dimension of said patch disposal slot coincides with a spacing between said third and fourth end walls, wherein each of said first and second sidewalls intersects with each of said first, second, third, and fourth end walls, and wherein a plurality of ribs extend from an interior surface of each of said first and second sidewalls such that said plurality of ribs are disposed with said internal chamber.

34. A transdermal patch disposal device comprising:
a containment comprising a recess on an exterior of said containment;
an internal chamber within said containment;
a patch disposal slot located within said recess, that extends completely through said containment, and that accesses said internal chamber; and
at least one transdermal patch disposed within said internal chamber, wherein said at least one transdermal patch comprises a first transdermal patch.

35. A transdermal patch disposal device comprising:
a containment;
an internal chamber within said containment;
a patch disposal slot that extends completely through said containment and that accesses said internal chamber;
first and second projections that extend within said internal chamber, wherein said patch disposal slot extends between said first and second projections, and wherein said first and second projections collectively define at least part of a depth of said patch disposal slot; and
at least one transdermal patch disposed within said internal chamber, wherein said at least one transdermal patch comprises a first transdermal patch.

36. A method of disposing transdermal patches using a containment that comprises an internal chamber and a patch disposal slot, said method comprising the steps of:
directing a first transdermal patch through said patch disposal slot;
changing said first transdermal patch from a first configuration into a second configuration that at least generally conforms with a length dimension of said patch disposal slot;
receiving said first transdermal patch within said internal chamber in response to said changing step; and
biasing said first transdermal patch from said second configuration back toward said first configuration when said first transdermal patch is within said internal chamber.
